(19) United States
(12) Patent Application Publication
(10) Pub. No.: US 2026/0216058 A1
(43) Pub. Date: Jul. 30, 2026

(54) TRACE ELEMENT COMPOSITION, PREPARATION METHOD THEREFOR, AND USE THEREOF

(71) Applicant: BEIJING ZANGWEIXINKANG PHARMACEUTICAL R&D CO., LTD., Changping Science Park, Beijing (CN)

(72) Inventors: Jun HU, Beijing (CN); Licun LU, Beijing (CN); Feng LIU, Beijing (CN); Yong ZHANG, Beijing (CN); Xue ZHANG, Beijing (CN)

(21) Appl. No.: 19/145,111

(22) PCT Filed: Jan. 2, 2024
(86) PCT No.: PCT/CN2024/070135

§ 371 Date: Jul. 1, 2025

(30) Foreign Application Priority Data
Jan. 3, 2023 (CN) 202310000643.5

(51) Int. Cl. A61K 9/00; A61K 9/08; A61K 31/295; A61K 31/30; A61K 31/315; A61K 33/04; A61K 33/16; A61K 33/18; A61K 33/26; A61K 33/30; A61K 33/32; A61K 33/34; A61K 47/02; A61P 7/08

(52) U.S. Cl. CPC A61K 9/0019; A61K 9/08; A61K 31/295; A61K 31/30; A61K 31/315; A61K 33/04; A61K 33/16; A61K 33/18; A61K 33/26; A61K 33/30; A61K 33/32; A61K 33/34; A61K 47/02; A61P 7/08

(57) ABSTRACT

a stable trace trace element composition, a preparation method therefor and the use thereof. The trace elementtrace element composition comprises a main material and an auxiliary material, the main material consists of medicinal organic acid salts of ferrum element, zinc element, copper element and manganese element, medicinal salts of fluorine element, iodine element, selenium element and molybdenum element, and medicinal organic acid salt or inorganic salt of chromium element, and the auxiliary material consists of water for injection and a pH regulator, the pH regulator being an inorganic acid. The pH value of the trace elementtrace element composition is 2.0-3.5. For per 10 mL of the trace elementtrace element composition, the amount of each trace elementtrace element satisfies (unit: μmol): 17.8-21.5 of ferrum, 40-100 of zinc, 4.7-9.6 of copper, 1 of manganese, 25-60 of fluorine, 0.5-1.2 of iodine, 0.75 to 1.27 of selenium, 0.2-0.26 of molybdenum and 0.2-0.4 of chromium.

### TECHNICAL FIELD

The present application relates to the field of pharmaceutical preparations, in particular to a trace element composition, a preparation method therefor and use thereof.

### BACKGROUND ART

Trace elements are essential micronutrients for an organism, which become a component of certain enzymes, hormones, and other substances in the body. Trace elements cannot be synthesized by the human body and need to be supplemented daily. Generally, trace elements can be supplemented through diet. In some special physiological and pathological conditions, such as pregnant women, or in cases of excessive catabolism (such as surgery, major trauma, burns), insufficient supply or abnormal loss or poor absorption (such as short bowel disease or Crohn's disease), there may be an increase in demand. To ensure normal physiological functions of the human body, trace element preparations are generally needed as supplements. For patients who require parenteral nutrition, especially those with poor digestion and absorption or unable to eat orally, it is necessary to supplement with appropriate amounts of various trace elements. In general, when used for patients with nutrient depletion or increased nutrient loss, the basic choice is to use commercially available multiple trace element parenteral nutrition preparations, and rarely to prepare single trace element preparations separately. In addition to the limited number of commercially available single trace element preparations, there are many types of trace element requirements, and the clinical preparation of single element preparations is complex, which can easily lead to preparation deviations and the risk of microbial contamination during the preparation process.

At present, the mainstream trace element products are mostly compound preparations, such as Multitrace-4 (containing trace elements zinc, copper, chromium and manganese) and Multitrace-5 (containing trace elements zinc, copper, chromium, manganese and selenium) in the North America market, Decan (containing 10 trace elements ferrum, zinc, copper, manganese, fluorine, cobalt, iodine, selenium, molybdenum and chromium) in the European market, Additrace, Tracutil and Addaven respectively containing 9 trace elements ferrum, zinc, copper, manganese, fluorine, iodine, selenium, molybdenum and chromium in the European market, as well as multiple trace element injections and multiple trace element injections (II) in the Chinese market, wherein the former contains 10 trace elements like Decan in the European market, and the latter (such as Addamel) contains 9 trace elements like Additrace in the European market with some differences in auxiliary material. Trace element injections for children include Neotrace-4 and Peditrace, and multiple trace element injections (I) are available on the domestic market, with the same formulation composition as Peditrace.

The compositions of multiple trace element injections available in the main market are shown in Table 1.

TABLE 1

| | Addamel | | | Addaven | | | Decan | | |
|---|---|---|---|---|---|---|---|---|---|
| Element/ | Elemental | | | Elemental | | | Elemental | | |
| trade name | form | Content | | form | Content | | form | Content | |
| ferrum | Ferric | 20 | μmol | Ferric | 20 | μmol | Ferrous | 17.90 | μmol |
| | chloride | | | chloride | | | gluconate | | |
| Zinc | Zinc | 100 | μmol | Zinc | 77 | μmol | Zinc | 153.0 | μmol |
| | chloride | | | chloride | | | gluconate | | |
| Copper | Copper | 20 | μmol | Copper | 6 | μmol | Copper | 7.550 | μmol |
| | chloride | | | chloride | | | gluconate | | |
| Manganese | Manganese | 5 | μmol | Manganese | 1 | μmol | Manganese | 3.640 | μmol |
| | chloride | | | chloride | | | gluconate | | |
| Fluorine | Sodium | 50 | μmol | Sodium | 50 | μmol | Sodium | 76.30 | μmol |
| | fluoride | | | fluoride | | | fluoride | | |
| Cobalt | — | — | | — | — | | Cobalt | 0.025 | μmol |
| | | | | | | | gluconate | | |
| Iodine | Potassium | 1 | μmol | Potassium | 1 | μmol | Sodium | 0.012 | μmol |
| | iodide | | | iodide | | | iodide | | |
| Selenium | Sodium | 0.4 | μmol | Sodium | 1 | μmol | Sodium | 0.887 | μmol |
| | selenite | | | selenite | | | selenite | | |
| Molybdenum | Sodium | 0.2 | μmol | Sodium | 0.2 | μmol | Ammonium | 0.261 | μmol |
| | molybdate | | | molybdate | | | heptamolybdate | | |
| Chromium | Chromium | 0.2 | μmol | Chromium | 0.2 | μmol | Chromium | 0.289 | μmol |
| | chloride | | | chloride | | | chloride | | |

| | | | |
|---|---|---|---|
| Strength | 10 mL | 10 mL | 40 mL |
| pH value | 2.0 to 2.6 | 2.5 | 4.0 to 4.5 |
| Auxiliary | Water for | Water for | Water |
| material | injection | injection | for injection |
| | as solvent | as solvent | as solvent |
| | Hydrochloric | Hydrochloric | Gluconic acid lactone |
| | acid as | acid as | as pH regulator |
| | pH regulator | pH regulator | and stabilizer |
| | Sorbitol as | Xylitol as | |
| | stabilizer | stabilizer | |

Wherein, the content refers to the dose of unit specification (count or bottle); “_” indicates not included; the pH value comes from the product instruction.

Gluconic acid lactone as pH regulator and stabilizer in DECAN products can undergo hydrolysis to produce gluconic acid in solution, and gluconic acid can be cyclized to form 6-membered cyclic gluconic acid delta-lactone and 5-membered cyclic gluconic acid gamma-lactone (Chromatography, Volume 8, Issue 4, 1990, Study on Hydrolysis of D-gluconic Acid Delta-Lactone Using High-Performance Liquid Chromatography). Literature (Nature, Aug. 24, 1963, 765-767) also shows that gluconic acid delta-lactone, gluconic acid gamma-lactone and gluconic acid can be converted to each other in solution. The mutual conversion and equilibrium of gluconic acid lactone and its solution chelate the relevant trace elements of DECAN product, playing the role of a stabilizer. Through process testing and detection, it has been found that in the formulation of DECAN product, in addition to introducing gluconic acid (gluconic acid lactone) into the main drug, the amount of gluconic acid ester added as an auxiliary material is about 2 mg/mL.

The formulation compositions of multiple trace element preparations are mostly composed of salts of various trace elements and auxiliary material. Representative components include inorganic salt-based trace elements and organic salt-based trace elements. As multiple trace elements form a compound formulation, the preparations need to meet the relevant requirements in clinic including safety, accurate dosage and the like. There are interactions between various trace elements and ions, which are prone to redox reactions and cause precipitation and solution discoloration. Different preparation processes also bring stability issues. At present, there are various technical methods to solve the problem of stable formulations, such as excessive feeding, adding stabilizers, making freeze-dried formulations, adding activated carbon and the like. In addition, technologies involving stabilizer injections, such as taurine, gluconic acid, xylitol, sorbitol, lactobionic acid, glycine, theanine, lysine, arginine, cysteine, methionine, trehalose, erythritol and the like, are commonly selected. The pH of injections with different formulation compositions varies. The prior art shows that the injections, wherein the trace elements in the formulations are mostly inorganic salt-based, have a relatively low pH, mostly between 2.0 and 2.6; and the injections, wherein the trace elements in the formulations are mostly organic salt-based, have a relatively high pH, mostly between 3.5 and 5.0, more between 4.0 and 4.5. Moreover, in general, for the products with a higher concentration of organic salt components, the pH regulator is an organic acid, such as gluconic acid (gluconic acid lactone).

### CONTENTS OF THE INVENTION

The present application provides a trace element composition, a preparation method therefor and use thereof, and aims to provide a trace element preparation with simple composition and good stability.

In a first aspect of the present application, there is provided a trace element composition, which comprises a main material and an auxiliary material, wherein the main material consists of medicinal organic acid salt of ferrum element, medicinal organic acid salt of zinc element, medicinal organic acid salt of copper element, medicinal organic acid salt of manganese element, medicinal salt of fluorine element, medicinal salt of iodine element, medicinal salt of selenium element, medicinal salt of molybdenum element, medicinal organic acid salt or medicinal inorganic salt of chromium element, the auxiliary material consists of water for injection and a pH regulator, wherein the pH regulator is an inorganic acid and an optional inorganic base, the pH value of the trace element composition is 2.0 to 3.5, and in each 10 mL of the trace element composition, each trace element in the main material meets the following dosage: 17.8 to 21.5 μmol of ferrum, 40 to 100 μmol of zinc, 4.7 to 9.6 μmol of copper, 1 μmol of manganese, 25 to 60 μmol of fluorine, 0.5 to 1.2 μmol of iodine, 0.75 to 1.27 μmol of selenium, 0.2 to 0.26 μmol of molybdenum and 0.2 to 0.4 μmol of chromium.

Further, in each 10 mL of the trace element composition, each trace element in the main material meets the following dosage: 18 to 21.5 μmol of ferrum, 50 to 100 μmol of zinc, 4.7 to 6 μmol of copper, 1 μmol of manganese, 50 μmol of fluorine, 0.5 to 1.0 μmol of iodine, 1.0 to 1.25 μmol of selenium, 0.2 μmol of molybdenum and 0.2 μmol of chromium.

Further, in each 10 ml of the trace element composition, each trace element in the main material meets the following dosage: 20 μmol of ferrum, 77 μmol of zinc, 4.7 μmol or 6 μmol of copper, 1 μmol of manganese, 50 μmol of fluorine, 1 μmol of iodine, 1 μmol of selenium, 0.2 μmol of molybdenum and 0.2 μmol of chromium.

Further, the pH value of the composition is 2.6 to 3.2.

Further, the inorganic acid is hydrochloric acid or sulfuric acid, preferably hydrochloric acid; the inorganic base is sodium hydroxide or potassium hydroxide, preferably sodium hydroxide.

Further, the organic acid salt of ferrum element is selected from any one of ferrous gluconate, ferrous L-aspartate, ferrous DL-aspartate and ferrous fumarate, the organic acid salt of zinc element is selected from any one of zinc gluconate, zinc L-aspartate and zinc DL-aspartate, the organic acid salt of copper element is selected from any one of copper gluconate, copper L-aspartate and copper DL-aspartate, the organic acid salt of manganese element is selected from any one of manganese gluconate, manganese L-aspartate and manganese DL-aspartate, the medicinal salt of fluorine element is sodium fluoride or potassium fluoride, the medicinal salt of iodine element is sodium iodide or potassium iodide, the medicinal salt of selenium element is sodium selenite or selenous acid, the medicinal salt of molybdenum element is sodium molybdate or ammonium heptamolybdate, the medicinal inorganic salt of chromium element is chromium chloride or chromium sulfate, and/or the organic acid salt of chromium element is selected from any one of chromium gluconate, chromium L-aspartate and chromium DL-aspartate.

Further, the main material consists of ferrous gluconate, zinc gluconate, copper gluconate, manganese gluconate, sodium fluoride, potassium iodide or sodium iodide, sodium selenite or sodium selenate, sodium molybdate and chromium chloride.

Further, the trace element composition is an injection; preferably, the inner wall of the packaging material of the injection is made of non-glass packaging material, and further preferably, the inner wall of the packaging material is made of polypropylene material.

According to another aspect of the present application, there is provided a preparation method of any trace element composition described above, comprising the steps of: dispersing salt of each trace element in water for injection, adjusting to a target pH value by using a pH regulator, filtering to obtain a filtrate, adding water to the filtrate to full volume, potting, and sterilizing, wherein sterilizing is preferably performed using a terminal sterilization process, the F₀ value of the terminal sterilization process is ≥8, and preferably ≥12.

Further, the packaging material in direct contact with the medicine during the potting is a non-glass packaging material, and preferably a polypropylene material.

Further, the trace element composition is prepared under the condition of being exposed to no higher than 500Lux illumination, preferably no higher than 300Lux illumination, and more preferably no higher than 100Lux illumination.

According to another aspect of the present application, there is provided the application of any trace element composition described above in a medicament for the prevention and treatment of trace element deficiency.

The composition of the present application is formed by replacing some inorganic salt elements in Addaven product. It is unexpectedly found that the composition formed after the replacement has better safety, and the preparation has better stability within the proposed pH range, without the need for additional stabilizers. The content of some elements (such as iodine) is more stable and does not require excessive feeding. Moreover, the trace element composition of the present application has significantly reduced product specification compared to Decan, making clinical use more convenient and safer.

### DESCRIPTION OF THE FIGURES

In order to more clearly illustrate the technical solutions of the examples of the present application, the drawings required to be used in the examples of the present application will be briefly described below, and it is obvious that the drawings described below are only some examples of the present application, and it is obvious for those skilled in the art that other drawings can be obtained according to the drawings without creative efforts.

FIG. 1 shows photographs of the samples from Example 1 with pH values of 2.0, 2.2, 2.4, 2.6, 2.9, 3.2, 3.5, 4.0, 4.3.

FIG. 2 shows photographs of the tubes Nos. 1-7 shaken well after sample addition in the in vitro hemolysis test on rabbit red blood cells in the examples.

FIG. 3 shows photographs of the tubes Nos. 1-7 3 hours after sample addition in the in vitro hemolysis test on rabbit red blood cells in the examples.

FIG. 4 shows photographs of the tubes Nos. 1-7 shaken well 3 hours after sample addition in the in vitro hemolysis test on rabbit red blood cells in the examples.

FIG. 5 shows a histopathological examination result of the animals with the control sample at the injection site on the administration side (left picture) and the control side (right picture) after 14 days of recovery.

FIG. 6 shows a histopathological examination result of the animals with the control sample at the proximal non-injection site on the administration side (left picture) and the control side (right picture) after 14 days of recovery.

FIG. 7 shows a histopathological examination result of the animals with the test sample at the injection site on the administration side (left picture) and the control side (right picture) after 14 days of recovery.

FIG. 8 shows a histopathological examination result of the animals with the test sample at the proximal non-injection site on the administration side (left picture) and the control side (right picture) after 14 days of recovery.

FIG. 9 shows a curve of the change in plasma copper concentration after administration of each formulation in the test groups in Table 29.

FIG. 10 shows a curve of the change in plasma manganese concentration after administration of each formulation in the test groups in Table 29.

FIG. 11 shows a curve of the change in plasma zinc concentration after administration of each formulation in the test groups in Table 29.

FIG. 12 shows a curve of the change in plasma selenium concentration after administration of each formulation in the test groups in Table 29

FIG. 13 shows the white blood cell number in the model animals before administration of each formulation in the test groups in Table 29.

FIG. 14 shows the white blood cell number in the model animals 4 days after administration of each formulation in the test groups in Table 29.

FIG. 15 shows a typical diagram of irritation after 4 consecutive days of tail vein administration of each formulation in the test groups in Table 29

### DETAILED DESCRIPTION OF SPECIFIC EMBODIMENTS

Embodiments of the present application will be described in further detail with reference to the drawings and examples. The following detailed description of the examples and the accompanying drawings are provided to illustrate the principle of the present application, but are not intended to limit the scope of the present application, i.e., the present application is not limited to the described examples.

As described in the background art of the present application, Addaven product employs hydrochloric acid to adjust the pH value, and xylitol as a stabilizer to improve the product stability. During the research of this product, it is found that although xylitol can improve the product stability, there is a significant loss of iodine content after sterilization. In order to ensure that the iodine content in the product reaches the set level, excessive addition of iodine is necessary. The reason for this is that the stability of Addaven product is insufficient. In order to solve this problem, the applicant of the present application has made various attempts in terms of pH value, amount of stabilizer added, type of stabilizer and the like, but none of them have improved the stability of iodine element. After experiencing the above failures, the present application attempts to replace the inorganic salts of ferrum, zinc, copper, and manganese elements with organic salts such as gluconate, and breaks conventional thinking by using inorganic acids instead of organic acids as pH regulators to adjust the pH value. This not only ensures the stability of the formed composition without excessive feeding of iodine elements, but also omits stabilizers, which further improves the safety and compliance of the formed trace element composition.

In a typical embodiment of the present application, there is provided a trace element composition, which comprises a main material and an auxiliary material, wherein the main material consists of medicinal organic acid salt of ferrum element, medicinal organic acid salt of zinc element, medicinal organic acid salt of copper element, medicinal organic acid salt of manganese element, medicinal salt of fluorine element, medicinal salt of iodine element, medicinal salt of selenium element, medicinal salt of molybdenum element, medicinal organic acid salt or medicinal inorganic salt of chromium element, the auxiliary material consists of water for injection and a pH regulator, wherein the pH regulator is an inorganic acid and an optional inorganic base, the pH value of the trace element composition is 2.0 to 3.5, and in each 10 mL of the trace element composition, each trace element in the main material meets the following dosage: 17.8 to 21.5 μmol of ferrum, 40 to 100 μmol of zinc, 4.7 to 9.6 μmol of copper, 1 μmol of manganese, 25 to 60 μmol of fluorine, 0.5 to 1.2 μmol of iodine, 0.75 to 1.27 μmol of selenium, 0.2 to 0.26 μmol of molybdenum and 0.2 to 0.4 μmol of chromium.

As described above, after replacing some inorganic salt elements in Addaven product, it is unexpectedly found that the product formed after the replacement has better safety, and the preparation has better stability within the proposed pH range, without the need for additional stabilizers. The content of some elements (such as iodine) is more stable and does not require excessive feeding. Moreover, the content of each trace element is set more reasonably, and the irritation to the applicator is lower during application And, it has a certain effect on reducing the number of white blood cells in rats caused by foreign bodies implanted in the back.

Although the trace element composition of the present application has similar or identical salts of common elements compared to Decan product, the product specification of the trace element composition of the present application is significantly reduced compared to Decan product after preparation into an injection solution. The product specification is reduced from 40 mL for Decan product to 10 mL for the present application. Taking 50,000 bottles in 40 mL specification as an example, the optimized product in 10 mL specification of the present application can be batched to 200,000 units, that is, the same volume of the product can reach a larger production lot (4 times), which greatly reduces the sterilization energy consumption of the unit product and the transportation volume of the drug, greatly reduces the production and transportation costs, and is convenient for clinical use. In addition, the pH regulator and pH value of the trace element composition in the present application are both different from those of Decan, and the trace element composition in the present application has good stability in the pH range of 2.0 to 3.5. Meanwhile, the zinc content of the trace element composition in the present application is somewhat reduced compared to Decan product. The preliminary safety tests show that the composition of the present invention has the advantage of fewer side effects.

In particular, the single dose specification and pH value of the injection formed by the trace element composition of the present application are different from those of products containing organic salts in the prior art, such as the main stream products DECAN, CN103340895 and CN104971074 with 40 mL specification and pH 3.5-5.0 (preferably 4.0-4.5) on the market. In addition to the effects of larger specification requiring larger packaging volume, the same lot requiring larger preparation production facilities, smaller commercial production lots with the same preparation amount, and larger storage and transportation volume and the like, the large specification of the injection also has an impact on the compatibility solution, and clinical compatibility application is relatively cumbersome. For example, as to the “DECAN” product described above, its drug instruction describes that one bottle of 40 mL specification product can be diluted into 250 ml of 0.9% sodium chloride injection solution, or 500 mL glucose injection solution or other suitable parenteral nutrition solutions. Commercially available 0.9% sodium chloride injection, glucose injection, or related parenteral nutrition solutions such as compound amino acid injection solutions have various packaging options, such as soft bags, glass bottles, and plastic bottles. Commercial products in different packaging have different headspace volumes, and the headspace volume of these products is sterile air or nitrogen. The headspace pressure is often basically the same as the external atmospheric pressure. For some compatibility solutions with lower headspace volume, it is difficult to continue add 40 ml of multiple trace elements therein, for example, the internal pressure of the compatibility solution as a closed packaging system is increased, which poses a risk of liquid leakage and inaccurate dosage, or the headspace volume is insufficient/difficult for the continuous addition of 40 mL, which brings difficulties to clinical applications. In addition, the increased volume brings about adverse effects on the compatibility solution itself in terms of, e.g., osmotic pressure and solution stability, and also increases the infusion time of the drug solution. The specification of the injection formed by the trace element composition of the present application is 10 mL, which can meet the dosage requirements and thus can better meet clinical applications. In addition, the pH regulator in the present application does not contain gluconic acid or gluconic acid lactone, avoiding the following problems that occur when using gluconic acid or gluconic acid lactone as a pH regulator: the pH value of the injection can only be at a relatively high level, such as 3.5-5.0 (preferably 4.0-4.5). Within this pH range, the stability of the product after sterilization is poor, and black precipitate may occur over time.

The research result also shows that even under the condition of a DECAN formulation, after the pH regulator and stabilizer are changed from gluconic acid lactone to hydrochloric acid, the pH value of the product of the DECAN formulation is obviously increased and the color of the solution is obviously darkened after sterilization, which indicates that the composition of the DECAN formulation is changed before and after sterilization, which may be due to that hydrochloric acid cannot play a role of complexing the elements like gluconic acid lactone, causing a change in the valence state of the elements due to oxidation-reduction reaction between the elements, so the safety is reduced.

When the selenium element is provided as selenious acid, inorganic acid and inorganic base can be simultaneously used as pH regulator.

In some embodiments of the present application, in each 10 ml of the trace element composition, each trace element in the main material meets the following dosage: 18 to 21.5 μmol of ferrum, 50 to 100 μmol of zinc, 4.7 to 6 μmol of copper, 1 μmol of manganese, 50 μmol of fluorine, 0.5 to 1.0 μmol of iodine, 1.0 to 1.25 μmol of selenium, 0.2 μmol of molybdenum and 0.2 μmol of chromium.

In some embodiments of the present application, in each 10 ml of the trace element composition, each trace element in the main material meets the following dosage: 20 μmol of ferrum, 77 μmol of zinc, 4.7 μmol or 6 μmol of copper, 1 μmol of manganese, 50 μmol of fluorine, 1 μmol of iodine, 1 μmol of selenium, 0.2 μmol of molybdenum and 0.2 μmol of chromium.

In some embodiments of the present application, in order to further improve the stability and safety of the trace element composition of the present application, the pH of the trace element composition is preferably 2.6 to 3.2.

The pH regulator used in the present application is an inorganic acid and optionally an inorganic base. In order to increase the adjustment efficiency of the pH regulator, in some embodiments, the inorganic acid is hydrochloric acid or sulfuric acid, preferably hydrochloric acid; the inorganic base is sodium hydroxide or potassium hydroxide, preferably sodium hydroxide. For example, sodium hydroxide or potassium hydroxide may be added as a pH regulator when selenium is provided as selenious acid. For safe use, the pH regulator may be diluted to a certain concentration in the process, such as 1M hydrochloric acid solution or 1M sodium hydroxide solution.

The medicinal organic salt of the trace elements ferrum, zinc, copper, manganese and chromium of the present application can be various common organic acid salts, such as gluconate, fumarate or amino acid salt, the organic acid salts of ferrum element being such as ferrous gluconate, ferrous L-aspartate, ferrous DL-aspartate and ferrous fumarate, the organic acid salts of zinc element being such as zinc gluconate, zinc L-aspartate and zinc DL-aspartate, the organic acid salts of copper element being such as copper gluconate, copper L-aspartate and copper DL-aspartate, the organic acid salts of manganese element being such as manganese gluconate, manganese L-aspartate and manganese DL-aspartate, the organic acid salts of chromium element being such as chromium gluconate, chromium L-aspartate and chromium DL-aspartate. The medicinal salts of the trace elements described above can be anhydrous or hydrate, preferably a solid existing form stable at normal temperature, such as related medicinal salts collected in pharmacopoeia at home and abroad.

The medicinal salts of the trace elements fluorine, iodine, molybdenum and chromium of the present application can be inorganic salts. The medicinal salt of fluorine can be sodium fluoride and potassium fluoride, the medicinal salt of iodine can be sodium iodide and potassium iodide, the medicinal salt of selenium can be sodium selenite and selenious acid, the medicinal salt of molybdenum can be sodium molybdate and ammonium heptamolybdate, the medicinal salt of chromium can be chromium chloride and chromium sulfate. The medicinal salts of the trace elements described above can be anhydrous or hydrate, preferably a solid existing form stable at normal temperature, such as related medicinal salts collected in pharmacopoeia at home and abroad.

In some embodiments, the main material consists of ferrous gluconate, zinc gluconate, copper gluconate, manganese gluconate, sodium fluoride, potassium iodide or sodium iodide, selenious acid or sodium selenite, sodium molybdate and chromium chloride. The ferrous gluconate, zinc gluconate, copper gluconate, manganese gluconate, sodium fluoride, potassium iodide or sodium iodide, selenious acid or sodium selenite, sodium molybdate and chromium chloride applied in the present application can be in different crystal water forms or anhydrous forms, these forms can be converted into element dosage of the present application, not affecting the effect of the present application. Selenious acid or sodium selenite also does not affect the actual effect of the present application, and after pH adjustment in the solution, they exist in the same form. Furthermore, as tested, when the main material consists of ferrous gluconate, zinc gluconate, copper gluconate, manganese gluconate, sodium fluoride, potassium iodide, sodium selenite, sodium molybdate and chromium chloride, the formed injection has better stability and safety.

In some embodiments of the present application, the components of the trace element composition described above are mixed to form an injection; preferably, the inner wall of the packaging material of the injection is made of non-glass packaging material, and further preferably, the inner wall of the packaging material is made of polypropylene material. Using polypropylene as the inner wall of the packaging material effectively avoids the influence of impurity elements caused by the packaging material. The above packaging material can be a polypropylene material as a whole.

In another typical embodiment of the present application, there is provided a preparation method of any trace element composition described above, comprising the steps of: dispersing salt of each trace element in water for injection, adjusting to a target pH value by using a pH regulator, filtering to obtain a filtrate, adding water to the filtrate to full volume, potting, and sterilizing.

The preparation method of the trace element composition of the present application is simple, and can be realized by adopting a conventional process.

In some embodiments, the above preparation method comprises the following steps:
dissolving and/or suspending salt of each trace element with water for injection, mixing uniformly, then continuing adding water for injection for complete dissolution, adjusting pH value, filtering, adding water for injection to full volume, potting by 10 mL per bottle, and sterilizing.

In some embodiments, the sterilizing is preferably performed using a terminal sterilization process, the F₀ value of the terminal sterilization process is ≥8, and preferably ≥12, and the sterilization temperature and time can be 115° C., 30 minutes, or 121° C., 8 to 20 minutes, preferably 121° C., 12 minutes and 121° C., 15 minutes. The salt of each trace element is dissolved or suspended in a plastic container, the dissolution or suspension depending on the amount of water added. Even if prepared as a suspension in the early stage, the salt can be dissolved well after being added to a mixing tank and diluted with a large amount of water for injection, without affecting the quality of the finished product.

The dissolving step can be mixing salts of multiple trace elements, dissolving them together in water, and then mixing uniformly; or dissolving salts of multiple trace elements in water respectively, and then mixing them together uniformly; or mixing salts of some trace elements, dissolving them together in water, while dissolving salts of rest trace elements in water respectively, and then mixing them together uniformly. No matter dissolving salts of multiple trace elements together in water, or dissolving salts of some trace elements in water, while dissolving salts of rest trace elements in water respectively, and then mixing them together, or dissolving salts of multiple trace elements in water respectively, and then mixing them together, under the condition that the amount of water added is enough (such as more than 70% of constant volume), the dissolving method has no influence on the quality of the final product of the composition. The enough amount of water added refers to the amount which at least can reach the condition that the salts of trace elements are well dissolved and the solution is clear. In general, in view of “trace”, even if for commercial production, the weighing of salts of some trace elements requires an analytical balance. For accurate weighing and feeding, to ensure the quality stability and consistency of the final product of the composition, the method of dissolving or suspending salts of trace elements respectively and then mixing them together is the preferred method. The amount of water for injection added for dissolving salts of multiple trace elements is preferably more than 70% of the total volume of the designed composition formulation, preferably 80% or more, and more preferably not less than 90%. To facilitate the dissolution rate, the temperature of the water for injection for the dissolution may be higher than room temperature, such as 30° C. to 70° C.

In the prior art, commercial products such as “DECAN” and “Addamel” are all packaged in glass materials. Although relevant literature shows that better glass packaging material can better ensure product quality, such as borosilicate glass, glass packaging has better air and water vapor isolation performance and better thermal conductivity than non-glass packaging (such as PP, PE), in order to avoid impurity elements brought by packaging material during the sterilization process, in some embodiments, the packaging material in direct contact with the medicine during the potting is a non-glass packaging material, such as cyclic olefin copolymer (COC), cyclic olefin polymer (COP), polypropylene (PP) materials, and preferably polypropylene material.

The preparation method of the present application has no special requirements on the illumination environment, or is carried out according to the dark requirement specified in the Pharmacopoeia of the People's Republic of China (2020), for example, avoiding direct sunlight. While direct sunlight illumination can reach 60,000-100,000 Lux, in order to improve the stability and safety of the product, the trace element composition of the present application is prepared by being exposed to no higher than 500Lux illumination, preferably no higher than 300Lux illumination, further preferably no higher than 100Lux illumination, and more preferably no higher than 75Lux illumination, whereby the stability of elements in the composition becomes better. In the production, it is more preferred that the preparation is carried out with a full-automatic production facility in a dark environment. The illumination refers to a lighting environment to which each intermediate product (e.g., an aqueous solution in the dissolving step, an intermediate after mixing, and a finished aqueous solution) is directly exposed in the preparation process of the trace element composition of the present application, and a light source required for the preparation may be a red light or a yellow light.

In a further typical embodiment of the present application, there is provided the use of any trace element composition described above in a medicament for the prevention and treatment of trace element deficiency, so as to meet the requirements of trace element parenteral nutrition. And, the medicament has better stability and safety on the basis of simplifying the product composition compared with Addaven products and Decan products.

The multi-trace element composition of the present invention can be used for preventing and treating trace element deficiency, particularly for patients who cannot take the composition orally and need intravenous nutrition. It meets the requirements of patients for basic to moderate trace elements, and can be used in multiple doses for patients with severe requirements, such as patients with severe burn.

The advantageous effects of the present application will be further described below with reference to examples and Comparative Examples. The following examples are merely illustrative of the technical solutions of the present application and should not be construed as limiting the scope of the claims of the present application.

The detection method involved in the examples of the present application, such as the method for detecting the content of each trace element, can be the same as the method of national standard revision draft for “Multi-trace element Injection (II)” re-published by the Chinese Pharmacopoeia Commission on May 12, 2014 (https://www.chp.org.cn/gjyjw/hxyp/494.jhtml). Due to the differences in formulation dosage between the present application and the multi-trace element injections (II), the specific amount of the test sample in the present application can be converted to be consistent or similar in terms of the content of the element to be tested, and within the range of the proposed linear regression equation, the element content can also be detected by ICP-Ms method, and iodine can also be detected by HPLC method. For other parameters such as pH value, clarity and color, characteristics, absorbance, visible foreign matter, insoluble particles and the like, please refer to the test methods and requirements in the Appendix of the Pharmacopoeia of the People's Republic of China. For the detection method of compatibility solution involved in the compatibility stability test, it can be performed with reference to the relevant compatibility solution quality standard method.

### Example 1

TABLE 2
Formulation (metered in 10 mL)

| | | Content of | |
|---|---|---|---|
| Composition | Effective element | effective element | |
| Ferrous gluconate | ferrum | 20 | μmol |
| Zinc gluconate | Zinc | 77 | μmol |
| Copper gluconate | Copper | 6 | μmol |
| Manganese gluconate | Manganese | 1 | μmol |
| Sodium fluoride | Fluorine | 50 | μmol |
| Potassium iodide | lodine | 1 | μmol |
| Sodium selenite | Selenium | 1 | μmol |
| Sodium molybdate | Molybdenum | 0.2 | μmol |
| Chromium chloride | Chromium | 0.2 | μmol |

| | | |
|---|---|---|
| Hydrochloric acid | pH regulator | Proper amount |

According to the above formulation, 20 L of product was prepared for each designed pH value formulation, in view of the theoretical quantity of 2,000 counts, the operation being carried out in a dark environment.

Ferrous gluconate, zinc gluconate, copper gluconate, manganese gluconate, sodium fluoride, sodium selenite, sodium molybdate, chromium chloride and potassium iodide were dissolved or suspended in plastic beakers with a proper amount of water for injection respectively, the resulting solutions or suspensions were added to a 20 L liquid preparation tank, the plastic beakers were rinsed with water for injection, the rinse water was merged into the liquid preparation tank, followed by stirring till complete dissolution, a 1M hydrochloric acid solution was added to adjust the pH value to be 2.0, 2.2, 2.4, 2.6, 2.9, 3.2, 3.5, 4.0 and 4.3 respectively, water for injection was added to full volume, followed by uniformly mixing with stirring, the resulting liquid medicine was filtered by a filter containing two 0.22 μm filter cartridges in series, potted in polypropylene ampoules with a loading amount of 10 mL/count, and sterilized at the temperature of 121° C. for 12 minutes.

### 1) Influence of pH Value

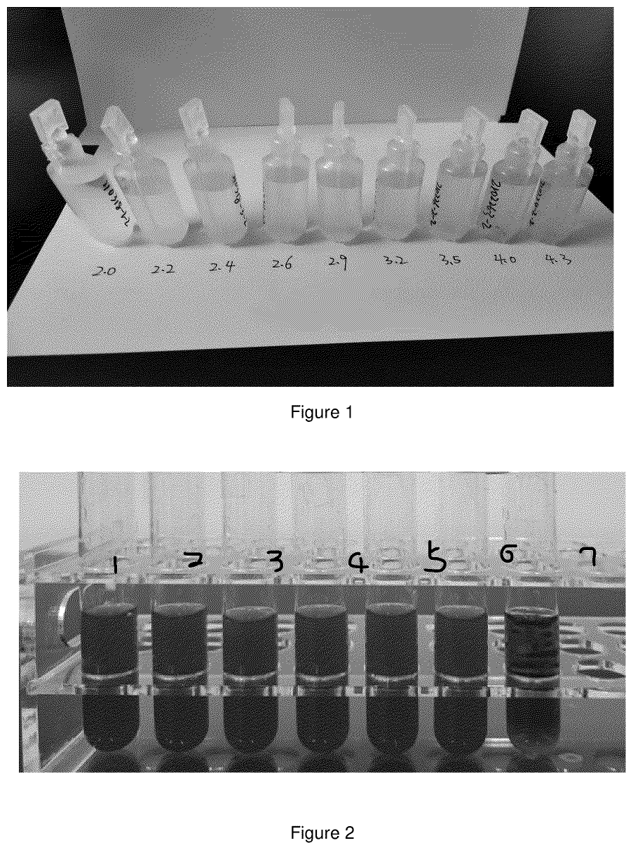

The representative sample is shown in FIG. 1. As can be seen from the trial production result of FIG. 1, as the pH value increases from left to right, the solution changes from colorless to yellow, and the color gradually becomes darker. Within the proposed pH range, the pH values of the solutions are basically consistent (±0.1) before and after sterilization; the detection results of the contents of elements (ferrum, zinc, copper, manganese, fluorine, selenium, molybdenum, chromium and iodine) in the samples with pH values of 2.0, 2.2, 2.4, 2.6, 2.9, 3.2 and 3.5 have no obvious change before and after sterilization, which are all within the range of 100%±3% of the theoretical amount of feeding, and the contents of copper and selenium elements in the samples with pH values of 4.0 and 4.3 are respectively reduced by 5.7% and 7.8% compared with those before sterilization.

### 2) Preliminary Thermal Stability Test of Formulation

The samples with pH values of 2.0, 2.2, 2.4, 2.6, 2.9, 3.2, 3.5, 4.0 and 4.3 were taken respectively, and placed at the temperature of 60° C. for 1 month, to examine the preliminary stability. A comparison was made in terms of solution clarity, color and pH value before and after the placement at the temperature of 60° C. for 1 month. The results are shown in Table 3.

TABLE 3
Preliminary thermal stability test results of formulation

| pH value of | Clarity and color of solution | | pH value of solution | |
|---|---|---|---|---|
| formulatiom | Day 0 | Day 30 | Day 0 | Day 30 |
| 2.0 | Colorless | Colorless and | 2.01 | 2.01 |
| | and clear | clear | | |
| 2.2 | Colorless | Colorless and | 2.23 | 2.23 |
| | and clear | clear | | |
| 2.4 | Colorless | Colorless and | 2.39 | 2.40 |
| | and clear | clear | | |
| 2.6 | Slightly | Slightly yellow | 2.63 | 2.63 |
| | yellow and | and clear | | |
| | clear | | | |
| 2.9 | Slightly | Slightly yellow | 2.95 | 2.96 |
| | yellow and | and clear | | |
| | clear | | | |
| 3.2 | Light yellow | Light yellow | 3.18 | 3.20 |
| | and clear | and clear | | |
| 3.5 | Yellow and | Yellow and | 3.52 | 3.53 |
| | clear | clear | | |
| 4.0 | Yellow and | Dark yellow | 4.02 | 3.87 |
| | clear | with black | | |
| | | precipitate | | |
| 4.3 | Yellow and | Dark yellow | 4.29 | 4.03 |
| | clear | with black | | |
| | | precipitate | | |

It can be seen from the above preliminary stability results that, at the same time that the color of the solution gradually darkens from colorless to yellow as the pH value increases, the color of the solution also gradually darkens as time passes, the color of the product solution with pH value of 3.5 or above obviously darkens, and the color of the sample solutions with pH value of 4.0 and pH value of 4.5 becomes dark yellow, with a decrease in pH value and black precipitate, indicating that the product stability is poor, and especially under the condition of accidental exposure to high temperature during long-term storage and transportation in summer, the product quality has risks.

The detection results of the contents of elements (ferrum, zinc, copper, manganese, fluorine, selenium, molybdenum, chromium and iodine) in the samples with pH values of 2.0, 2.2, 2.4, 2.6, 2.9, 3.2 and 3.5 have no obvious change after the placement under the condition of 60° C. for 1 month, which are all within the range of 100%+3% of the theoretical amount of feeding. The characteristics of the samples with pH values of 4.0 and 4.3 do not meet the requirements due to the occurrence of black precipitate, so the contents of elements are not further detected.

### 3) Photostability Test

The sample with pH value of 2.9 was taken as an example, and placed under 500Lux, 300Lux, 100Lux and 50 Lux respectively, for the detection of absorbance (color) at 325 nm and 420 nm. The absorbance was detected using water as a blank according to ultraviolet-visible spectrophotometry (Part IV General Chapter 0401 of Pharmacopoeia of the People's Republic of China (2020)) at the wavelengths of 325 nm and 420 nm. The absorbance results are shown in Table 4.

TABLE 4
Photostability test results

| Illuminance | 500 Lux | | 300 Lux | | 100 Lux | | 50 Lux | |
|---|---|---|---|---|---|---|---|---|
| Absorbance | 325 nm | 420 nm | 325 nm | 420 nm | 325 nm | 420 nm | 325 nm | 420 nm |
| 0 hour | 0.545 | 0.074 | 0.545 | 0.074 | 0.545 | 0.074 | 0.545 | 0.074 |
| 2 hours | 0.540 | 0.074 | 0.544 | 0.073 | 0.545 | 0.074 | 0.546 | 0.074 |
| 4 hours | 0.532 | 0.067 | 0.543 | 0.073 | 0.545 | 0.073 | 0.545 | 0.074 |
| 8 hours | 0.521 | 0.060 | 0.539 | 0.071 | 0.544 | 0.074 | 0.544 | 0.074 |
| 12 hours | 0.496 | 0.052 | 0.535 | 0.068 | 0.544 | 0.073 | 0.545 | 0.074 |

As can be seen from the photostability results in Table 4, the absorbance has no large change within 4 hours under 300Lux, which can meet the general commercial production requirements, and the absorbance is stable under 100Lux, which can meet the process requirements of mass production.

### Example 2 Commercial Formulation and Process

The formulation was the same as in Example 1. According to the formulation, 350 L of product was prepared in view of a quantity of 35,000 counts, the operation being carried out in a dark environment.

Preparation of 1M hydrochloric acid solution: 1000 mL of hydrochloric acid was diluted with water for injection into a 1M hydrochloric acid solution, which was uniformly mixed for later use.

Water for injection was added to a liquid preparation tank, while starting and keeping it in a stirring state, ferrous gluconate, zinc gluconate, copper gluconate, manganese gluconate, sodium fluoride were dissolved or suspended in a plastic beaker with a proper amount of water for injection respectively, the resulting solutions or suspensions were added to the liquid preparation tank, followed by dissolution with stirring, the plastic beaker was rinsed with a proper amount of water for injection, and the rinse water was merged into the liquid preparation tank; sodium selenite, sodium molybdate, chromium chloride and potassium iodide were dissolved in plastic beakers with a proper amount of water for injection respectively, the resulting solutions were added to the liquid preparation tank, the plastic beakers were rinsed with water for injection, the rinse water was merged into the liquid preparation tank, a 1M hydrochloric acid solution was added to adjust the pH value to be 2.8~3.0, water for injection was added to full volume, an intermediate solution was taken for detection (characteristics, pH value, bacterial endotoxin and content of each trace element), and the resulting liquid medicine was filtered by a filter containing two 0.22 μm filter cartridges in series to a buffer tank before a potting machine, ready for potting.

A BFS plastic ampoule potting machine conveyed polypropylene resin to a hopper of a BFS extruder through a vacuum material suction device, the liquid medicine in the buffer tank was pressed into a liquid separation device according to a specified program, ampoule manufacture and potting were started, with a loading amount of 10 ml/count, the potted product was conveyed to a sterilization procedure through a conveying belt, and sterilized at the temperature of 121° C. for 12 minutes.

Vacuum leak detection was carried out in a water bath sterilization leak detection cabinet according to a specified program, using a high-pressure electronic leak detection machine, to reject unqualified products. Qualified products underwent lamp detection, and packaged.

The detection results of the contents of elements (ferrum, zinc, copper, manganese, fluorine, selenium, molybdenum, chromium and iodine) in the sample have no obvious change before and after the sterilization, which are all within the range of 100%±3% of the theoretical amount of feeding. The characteristics and pH value both have no change, and both meet the requirements.

### Example 3

The formulation was the same as that of Example 1 except for that the content of effective element in the copper gluconate was changed from 6 μmol to 4.7 μmol.

According to the formulation in this example, 10 L of product was prepared in view of the theoretical quantity of 1,000 counts, the operation being carried out in a dark environment. The preparation method was the same as that of Example 2.

The detection results of the contents of elements (ferrum, zinc, copper, manganese, fluorine, selenium, molybdenum, chromium and iodine) in the sample have no obvious change before and after the sterilization, which are all within the range of 100%±3% of the theoretical amount of feeding. The characteristics and pH value both have no change, and both meet the requirements.

### Example 4

The formulation was the same as that of Example 1 except for that the content of effective element in the copper gluconate was changed from 6 μmol to 4.7 μmol, sodium iodide was used in place of potassium iodide, and selenious acid was used in place of sodium selenite.

According to the formulation in this example, 10 L of product was prepared in view of the theoretical quantity of 1,000 counts, the operation being carried out in a dark environment. The preparation method was the same as that of Example 2 except for that the pH regulator was a 1M hydrochloric acid solution and a 1M sodium hydroxide solution.

The detection results of the contents of elements (ferrum, zinc, copper, manganese, fluorine, selenium, molybdenum, chromium and iodine) in the sample have no obvious change before and after the sterilization, which are all within the range of 100%±3% of the theoretical amount of feeding. The characteristics and pH value both have no change, and both meet the requirements.

### Example 5

TABLE 5
Formulation (metered in 10 mL)

| | | Content of | |
|---|---|---|---|
| Composition | Effective element | effective element | |
| Ferrous gluconate | Ferrum | 18 | μmol |
| Zinc gluconate | Zinc | 40 | μmol |
| Copper gluconate | Copper | 4.7 | μmol |
| Manganese gluconate | Manganese | 1 | μmol |
| Sodium fluoride | Fluorine | 50 | μmol |
| Potassium iodide | lodine | 0.5 | μmol |
| Sodium selenite | Selenium | 1 | μmol |
| Sodium molybdate | Molybdenum | 0.2 | μmol |
| Chromium chloride | Chromium | 0.2 | μmol |

| | | |
|---|---|---|
| Hydrochloric acid | pH regulator | Proper amount |

According to the above main drug formulation, 10 L of product was prepared in view of the theoretical quantity of 1,000 counts, the operation being carried out in a dark environment.

Ferrous gluconate, zinc gluconate, copper gluconate, manganese gluconate, sodium fluoride, sodium selenite, sodium molybdate, chromium chloride and potassium iodide were dissolved in plastic beakers with a proper amount of water for injection respectively, the resulting solutions were added to a liquid preparation tank, the plastic beakers were rinsed with water for injection, the rinse water was merged into the liquid preparation tank, a 1M hydrochloric acid solution was added to adjust the pH value to be 2.8~3.0, water for injection was added to full volume, followed by uniformly mixing with stirring, the resulting liquid medicine was filtered by a filter containing two 0.22 μm filter cartridges in series, potted in polypropylene ampoules with a loading amount of 10 mL/count, and sterilized at the temperature of 121° C. for 12 minutes. The product underwent lamp detection, and packaged.

The sterilized product was placed at 60° C. for 30 days. The quality indexes of the product including solution clarity, color, pH value, content of each component and the like have no obvious change before and after the sterilization as well as before and after the placement of the product at 60° C. for 30 days. The product has good quality stability.

### Example 6

TABLE 6
Formulation (metered in 10 mL)

| | | Content of effective | |
|---|---|---|---|
| Composition | Effective element | element | |
| Ferrous gluconate | Ferrum | 21.5 | μmol |
| Zinc gluconate | Zinc | 100 | μmol |
| Copper gluconate | Copper | 9.6 | μmol |
| Manganese gluconate | Manganese | 1 | μmol |
| Sodium fluoride | Fluorine | 50 | μmol |
| Potassium iodide | lodine | 1.2 | μmol |
| Sodium selenite | Selenium | 1.25 | μmol |
| Sodium molybdate | Molybdenum | 0.2 | μmol |
| Chromium chloride | Chromium | 0.2 | μmol |

| | | |
|---|---|---|
| Hydrochloric acid | pH regulator | Proper amount |

According to the above main drug formulation, 10 L of product was prepared in view of the theoretical quantity of 1,000 counts, the operation being carried out in a dark environment.

Ferrous gluconate, zinc gluconate, copper gluconate, manganese gluconate, sodium fluoride, sodium selenite, sodium molybdate, chromium chloride and potassium iodide were dissolved in plastic beakers with a proper amount of water for injection respectively, the resulting solutions were added to a liquid preparation tank, the plastic beakers were rinsed with water for injection, the rinse water was merged into the liquid preparation tank, a 1M hydrochloric acid solution was added to adjust the pH value to be 2.8~3.0, water for injection was added to full volume, followed by uniformly mixing with stirring, the resulting liquid medicine was filtered by a filter containing two 0.22 μm filter cartridges in series, potted in polypropylene ampoules with a loading amount of 10 mL/count, and sterilized at the temperature of 121° C. for 12 minutes. The product underwent lamp detection, and packaged.

The sterilized product was placed at 60° C. for 30 days. The quality indexes of the product including solution clarity, color, pH value, content of each component and the like have no significant change before and after the sterilization as well as before and after the placement of the product at 60° C. for 30 days. The product has good quality stability.

### Example 7

TABLE 7
Formulation (metered in 10 mL)

| | | Content of | |
|---|---|---|---|
| Composition | Effective element | effective element | |
| Ferrous gluconate | Ferrum | 20 | μmol |
| Zinc gluconate | Zinc | 77 | μmol |
| Copper gluconate | Copper | 6 | μmol |
| Manganese gluconate | Manganese | 1 | μmol |
| Sodium fluoride | Fluorine | 50 | μmol |
| Sodium iodide | lodine | 1 | μmol |
| Selenious acid | Selenium | 1 | μmol |
| Sodium molybdate | Molybdenum | 0.2 | μmol |
| Chromium chloride | Chromium | 0.2 | μmol |

| | | |
|---|---|---|
| Hydrochloric acid | pH regulator | Proper amount |

According to the above main drug formulation, 10 L of product was prepared in view of the theoretical quantity of 1,000 counts, the operation being carried out in a dark environment.

Ferrous gluconate, zinc gluconate, copper gluconate, manganese gluconate, sodium fluoride, selenious acid, sodium molybdate, chromium chloride and sodium iodide were dissolved in plastic beakers with a proper amount of water for injection respectively, the resulting solutions were added to a liquid preparation tank, the plastic beakers were rinsed with water for injection, the rinse water was merged into the liquid preparation tank, a 1M hydrochloric acid solution or sodium hydroxide solution was added to adjust the pH value to be 2.8~3.0, water for injection was added to full volume, followed by uniformly mixing with stirring, the resulting liquid medicine was filtered by a filter containing two 0.22 μm filter cartridges in series, potted in polypropylene ampoules with a loading amount of 10 mL/count, and sterilized at the temperature of 121° C. for 12 minutes. The product underwent lamp detection, and packaged.

The sterilized product was placed at 60° C. for 30 days. The quality indexes of the product including solution clarity, color, pH value, content of each component and the like have no obvious change before and after the sterilization as well as before and after the placement of the product at 60° C. for 30 days. The product has good quality stability.

### Example 8

The formulation and preparation method were the same as those of Example 2 except for that the packaging material was a low borosilicate glass ampoule and a medium borosilicate glass ampoule. 10 L of products were each prepared in view of the theoretical quantity of 1,000 counts.

The above samples of Example 7 and Example 8 were subjected to the detection of some impurity elements, using inductively coupled plasma atomic emission spectrometry (ICP) and inductively coupled plasma mass spectrometry (ICP-Ms). The results are shown in Table 8.

TABLE 8
Detection results of some impurity elements

| | Low borosilicate | | Medium borosilicate | | Polypropylene | |
|---|---|---|---|---|---|---|
| | glass ampoule | | glass ampoule of | | ampoule of | |
| | of Example 8 | | Example 8 | | Example 7 | |
| Impurity | Before | After | Before | After | Before | After |
| element | sterilization | sterilization | sterilization | sterilization | sterilization | sterilization |
| B | 1.51 | 3.44 | 1.04 | 2.26 | 0.01 | 0.01 |
| (μg/mL) | | | | | | |
| Si | 15.12 | 62.87 | 9.75 | 32.89 | 0.21 | 0.22 |
| (μg/mL) | | | | | | |
| Ba | 1.22 | 1.96 | 0.12 | 0.19 | 0.03 | 0.03 |
| (μg/mL) | | | | | | |
| Al | 1.13 | 10.58 | 0.62 | 9.47 | 0.01 | 0.01 |
| (μg/mL) | | | | | | |

It can be seen that, with the use of the glass ampoule, the content of impurity elements is obviously higher, and the content of impurity elements is obviously increased after the sterilization; whereas, the product with the use of polypropylene ampoule has a very low level of impurity elements, with no change before and after the sterilization, wherein the related impurity elements should be introduced as raw materials, and the packaging material has no influence on the impurity elements.

DEACN product (Lot No.: 9901563, source: Laboratoire AGUETTANT) was subjected to the above-mentioned impurity element detection. The results show that the content of B, Si, Ba and Al is respectively 8.92 μg/mL, 0.56 μg/mL, 29.43 μg/mL and 7.87 μg/mL. The content of Al in each bottle is nearly 315 μg in terms of 40 mL specification (basic daily dosage), which exceeds the risk control dose (4-5 μg/kg) for aluminum toxicity based on 50 kg adult body weight.

It can be seen that the above impurity elements bring safety risks. Most of the time, parenteral nutrition supplements of such preparations last for a long time, and patients who receive parenteral nutrition are affected by underlying diseases and have poor physical health. In addition, long-term medication often leads to liver and kidney function damage and deficiencies. The USP (United States Pharmacopeia) clearly states that for patients with kidney function damage, including premature newborns, when the cumulative amount of aluminum absorbed through the intestine exceeds 4-5 μg/kg per day, central nervous system and bone toxicity may occur, and at lower doses, aluminum loading may occur in tissues. Aluminum levels in patients with kidney function damage, who receive long-term parenteral nutrition, should be monitored regularly to prevent aluminum poisoning.

As can be seen from the above test results, when using polypropylene as the packaging material, the trace element composition injection of the present application has a significantly lower level of impurity elements, and the influence of the impurity elements contained therein on the safety is negligible.

### Example 9

TABLE 9
Formulation (metered in 10 mL)

| | | Content of | |
|---|---|---|---|
| Composition | Effective element | effective element | |
| Ferrous gluconate | Ferrum | 20 | μmol |
| Zinc gluconate | Zinc | 50 | μmol |
| Copper gluconate | Copper | 6 | μmol |
| Manganese gluconate | Manganese | 1 | μmol |
| Sodium fluoride | Fluorine | 50 | μmol |
| Potassium iodide | lodine | 1 | μmol |
| Sodium selenite | Selenium | 1 | μmol |
| Sodium molybdate | Molybdenum | 0.2 | μmol |
| Chromium chloride | Chromium | 0.2 | μmol |

| | | |
|---|---|---|
| Hydrochloric acid | pH regulator | Proper amount |

According to the above main drug formulation, 10 L of product was prepared in view of the theoretical quantity of 1,000 counts, the operation being carried out in a dark environment.

Ferrous gluconate, zinc gluconate, copper gluconate, manganese gluconate, sodium fluoride, selenious acid, sodium molybdate, chromium chloride and sodium iodide were dissolved in plastic beakers with a proper amount of water for injection respectively, the resulting solutions were added to a liquid preparation tank, the plastic beakers were rinsed with water for injection, the rinse water was merged into the liquid preparation tank, a 1M hydrochloric acid solution was added to adjust the pH value to be 2.8~3.0, water for injection was added to full volume, followed by uniformly mixing with stirring, the resulting liquid medicine was filtered by a filter containing two 0.22 μm filter cartridges in series, potted in polypropylene ampoules with a loading amount of 10 mL/count, and sterilized at the temperature of 121° C. for 12 minutes. The product underwent lamp detection, and packaged.

The sterilized product was placed at 60° C. for 30 days. The quality indexes of the product including solution clarity, color, pH value, content of each component and the like have no obvious change before and after the sterilization as well as before and after the placement of the product at 60° C. for 30 days. The product has good quality stability.

### Comparative Example 1

The dose of zinc gluconate was increased to 153 μmol with reference to the formulation of DECAN.

TABLE 10
Formulation (metered in 10 mL)

| | | Content of | |
|---|---|---|---|
| Composition | Effective element | effective element | |
| Ferrous gluconate | Ferrum | 20 | μmol |
| Zinc gluconate | Zinc | 153 | μmol |
| Copper gluconate | Copper | 6 | μmol |
| Manganese gluconate | Manganese | 1 | μmol |
| Sodium fluoride | Fluorine | 50 | μmol |
| Potassium iodide | lodine | 1 | μmol |
| Sodium selenite | Selenium | 1 | μmol |
| Sodium molybdate | Molybdenum | 0.2 | μmol |
| Chromium chloride | Chromium | 0.2 | μmol |

| | | |
|---|---|---|
| Hydrochloric acid | pH regulator | Proper amount |

According to the above formulation, 10 L of product was prepared in view of the theoretical quantity of 1,000 counts, the operation being carried out in a dark environment. The preparation method was the same as that of Example 4.

The detection results of the contents of elements (ferrum, zinc, copper, manganese, fluorine, selenium, molybdenum, chromium and iodine) in the sample have no obvious change before and after the sterilization, which are all within the range of 100%+3% of the theoretical amount of feeding. The characteristics and pH value both have no change. A comparison between the samples of Comparative Example 1 and Example 9 shows that the solution color of the sample of Comparative Example 1 is darker than that of Example 9 before and after the sterilization, indicating that a higher content of zinc gluconate may affect the color of the product.

### Comparative Example 2

TABLE 11
Formulation (metered in 10 mL)

| | | Content of | |
|---|---|---|---|
| Composition | Effective element | effective element | |
| Ferrous gluconate | Ferrum | 20 | μmol |
| Zinc gluconate | Zinc | 77 | μmol |
| Copper gluconate | Copper | 6 | μmol |
| Manganese gluconate | Manganese | 1 | μmol |
| Sodium fluoride | Fluorine | 50 | μmol |
| Potassium iodide | lodine | 1 | μmol |
| Sodium selenite | Selenium | 1 | μmol |
| Sodium molybdate | Molybdenum | 0.2 | μmol |
| Chromium chloride | Chromium | 0.2 | μmol |

| | | |
|---|---|---|
| Gluconic acid lactone | pH regulator | Proper amount |

Remark: the pH regulator gluconic acid lactone, after being dissolved in water, forms gluconic acid lactone solution, namely gluconic acid solution.

According to the above formulation, 10 L of product was prepared for each designed pH value formulation, in view of the theoretical quantity of 1,000 counts, the operation being carried out in a dark environment.

Ferrous gluconate, zinc gluconate, copper gluconate, manganese gluconate, sodium fluoride, sodium selenite, sodium molybdate, chromium chloride and potassium iodide were dissolved or suspended in plastic beakers with a proper amount of water for injection respectively, the resulting solutions or suspensions were added to a 10 L liquid preparation tank, the plastic beakers were rinsed with water for injection, the rinse water was merged into the liquid preparation tank, followed by stirring till complete dissolution, gluconic acid lactone solution was added with an intention to adjust the pH value to be 2.0, 2.2, 2.4, 2.6, 2.9, 3.2, 3.5, 4.0 and 4.3 respectively, water for injection was added to full volume, followed by uniformly mixing with stirring. The subsequent filtering, potting and sterilizing processes were the same as those of Example 4 (the resulting liquid medicine was filtered by a filter containing two 0.22 μm filter cartridges in series, potted in polypropylene ampoules with a loading amount of 10 mL/count, and sterilized at the temperature of 121° C. for 12 minutes). The feasibility under the conditions of different pH values was tested.

The test results show that it is difficult to realize the samples with pH values of less than 3.5 by using gluconic acid lactone to adjust the pH value. In addition to the pH value of 3.5, the pH value of the solution reduces little by further adding a large amount of gluconic acid lactone, and the pH value of the solution cannot be further reduced after the pH value is reduced to 3.3. Even if the unit solution specification exceeds the designed 10 mL due to the further addition, the target lower pH value cannot be achieved. That is, on one hand, the above formulation requires a large amount of gluconic acid lactone, on the other hand, it is difficult to adjust to the target lower pH value by using gluconic acid lactone, and thus it is difficult to meet the commercial process requirements of products with pH values of 2.0, 2.2, 2.4, 2.6, 2.9 and 3.2. And, the sterilized samples with pH values of 3.5, 4.0 and 4.5 all have black precipitate after being placed at 60° C. for 1 month.

The inventors also tried the effect when the dose of DECAN unit formulation was reduced from 40 mL to 10 mL, like that in the example of the present application. The results are consistent with the above, that is, it is difficult to realize the samples with pH values of less than 3.5 by using gluconic acid lactone to adjust the pH value, and thus it is difficult to realize the commercial process requirements of products with pH values of 2.0, 2.2, 2.4, 2.6, 2.9 and 3.2. And, the sterilized samples with pH values of 3.5, 4.0 and 4.5 all have black precipitate after being placed at 60° C. for less than 1 month.

### Comparative Example 3

Prepared according to DECAN formulation (40 mL/bottle).

TABLE 12
Formulation (metered in 40 mL)

| | | Content of effective | |
|---|---|---|---|
| Composition | Effective element | element (per 40 mL) | |
| Ferrous gluconate | Ferrum | 17.90 | μmol |
| Zinc gluconate | Zinc | 153.0 | μmol |
| Copper gluconate | Copper | 7.55 | μmol |
| Manganese gluconate | Manganese | 3.64 | μmol |
| Sodium fluoride | Fluorine | 76.30 | μmol |
| Cobalt gluconate | Cobalt | 0.025 | μmol |
| Sodium iodide | lodine | 0.012 | μmol |
| Sodium selenite | Selenium | 0.887 | μmol |
| Ammonium heptamolybdate | Molybdenum | 0.261 | μmol |
| tetrahydrate | | | |
| Chromium chloride | Chromium | 0.289 | μmol |

The preparation was carried out in an amount of 500 times (20 L) of the above formulation, the operation being carried out in a dark environment:
(1) Sodium fluoride, ferrous gluconate, zinc gluconate, copper gluconate, manganese gluconate, sodium iodide, cobalt gluconate, ammonium heptamolybdate tetrahydrate, sodium selenite and chromium chloride were dissolved in plastic beakers with a proper amount of water for injection respectively, the resulting solutions were added to a liquid preparation tank, the plastic beakers were rinsed with water for injection, the rinse water was merged into the liquid preparation tank, a 1M hydrochloric acid solution was added to adjust the pH value to be 4.1~4.4, water for injection was added to full volume, followed by uniformly mixing with stirring, the resulting liquid medicine was filtered by a filter containing two 0.22 μm filter cartridges in series, potted at 40 mL/bottle, and sterilized at the temperature of 121° C. for 12 minutes.
(2) Sodium fluoride, ferrous gluconate, zinc gluconate, copper gluconate, manganese gluconate, sodium iodide, cobalt gluconate, ammonium heptamolybdate tetrahydrate, sodium selenite and chromium chloride were dissolved in a plastic beaker with a proper amount of water for injection respectively, the resulting solutions were added to a liquid preparation tank, the plastic beaker was rinsed with water for injection, the rinse water was merged into the liquid preparation tank, a 20% gluconic acid lactone solution was added to adjust the pH value to be 4.1~4.4, water for injection was added to full volume, followed by uniformly mixing with stirring, the resulting liquid medicine was filtered by a filter containing two 0.22 μm filter cartridges in series, potted at 40 mL/bottle, and sterilized at the temperature of 121° C. for 12 minutes.

The above products were compared in terms of characteristics, pH value and the like before and after the sterilization, showing that:

The pH value of the product solution (1) using hydrochloric acid to adjust the pH value is 4.32 before the sterilization, and 4.60 after the sterilization, indicating an obvious increase of the pH value; the color of the solution is a light yellow clear liquid (less than a yellow green No. 3 standard colorimetric solution) before the sterilization, and obviously darkens (close to a yellow green No. 5 standard colorimetric solution) after the sterilization;

In the case of, the pH value of the product solution (2) using gluconic acid lactone solution to adjust the pH value is 4.32 before the sterilization, and 4.30 after the sterilization, without obvious change in the pH value; the color of the solution is a light yellow clear liquid (less than a yellow green No. 3 standard colorimetric solution) before and after the sterilization, without obvious change in the color of the solution. It indicates that, when the pH regulator is changed from gluconic acid (lactone) to hydrochloric acid under the condition of DECAN formulation, the solution system is unstable after the solution is sterilized, the pH value is significantly increased and the color significantly darkens, and the pH value after the sterilization exceeds the pH value range (4.0~4.5) set by the DECAN standard.

### Comparative Example 4

With reference to Addaven product, the composition of main drug components in terms of trace elements is as follows (metered in 10 mL specification):

TABLE 13
Formulation (metered in 10 mL)

| Main drug components | Trace elements and content thereof | |
|---|---|---|
| Chromium chloride (CrCl₃•6H₂O) | Chromium | 0.20 μmol |
| Copper chloride (CuCl₂•2H₂O) | Copper | 6.0 μmol |
| Ferric chloride (FeCl₃•6H₂O) | Ferrum | 20 μmol |
| Manganese chloride | Manganese | 1.0 μmol |
| (MnCl₂•4H₂O) | | |
| Potassium iodide (KI) | Iodine | 1.0 μmol |
| Sodium fluoride (NaF) | Fluorine | 50 μmol |
| Sodium molybdate | Molybdenum | 0.20 μmol |
| (Na₂MoO₄•2H₂O) | | |
| Sodium selenite (NazSeO₃) | Selenium | 1.0 μmol |
| Zinc chloride (ZnCl₂) | Zinc | 77 μmol |

According to the above main drug formulation, 10 L of product was prepared in view of the theoretical quantity of 1,000 counts, the operation being carried out in a dark environment.

Ferric chloride, zinc chloride, copper chloride, manganese chloride, sodium fluoride, sodium selenite, sodium molybdate, chromium chloride and potassium iodide were dissolved or suspended in plastic beakers with a proper amount of water for injection respectively, the resulting solutions or suspensions were added to a 10 L liquid preparation tank, the plastic beakers were rinsed with water for injection, the rinse water was merged into the liquid preparation tank, followed by stirring till complete dissolution, a 1M hydrochloric acid solution was added to adjust the pH value to be 1.8, 2.0, 2.2, 2.4 and 2.6, water for injection was added to full volume, followed by uniformly mixing with stirring, the resulting liquid medicine was filtered by a filter containing two 0.22 μm filter cartridges in series, potted in polypropylene ampoules with a loading amount of 10 mL/count, and sterilized at the temperature of 121° C. for 12 minutes.

Results: the samples with pH values of 2.4 and 2.6 are clear liquids almost colorless to light yellow before the sterilization, and yellow turbid liquids after the sterilization, indicating that, for the sample without containing xylitol as a stabilizer, the product after terminal sterilization does not meet the requirements; the samples with pH values of 1.8, 2.0, 2.2 are all almost colorless clear liquids before and after the sterilization, indicating that, after further pH reduction, the Addaven product formulation without containing xylitol can be prepared into a clear solution sample.

### Comparative Example 5

With reference to Addaven product, a xylitol stabilizer was added. Being identical with the process of the above formulation, ferric chloride, zinc chloride, copper chloride, manganese chloride, sodium fluoride, sodium selenite, sodium molybdate, chromium chloride and potassium iodide were dissolved or suspended in plastic beakers with a proper amount of water for injection respectively, the resulting solutions or suspensions were added to a 10 L liquid preparation tank in which xylitol was already dissolved, the plastic beakers were rinsed with water for injection, the rinse water was merged into the liquid preparation tank, followed by stirring till complete dissolution, a 1M hydrochloric acid solution was added to adjust the pH value to be 2.4~2.6, water for injection was added to full volume, followed by uniformly mixing with stirring, the resulting liquid medicine was filtered by a filter containing two 0.22 μm filter cartridges in series, potted in polypropylene ampoules with a loading amount of 10 mL/count, and sterilized at the temperature of 121° C. for 12 minutes. The product is a clear liquid almost colorless to light yellow before and after the sterilization.

In addition, the samples of Comparative Examples 4 and 5 were tested to obtain the results shown in Table 14, taking the amount of the formulation as 100%.

TABLE 14

| | Comparative Example 4 | | Comparative Example 5 | |
|---|---|---|---|---|
| | Detection index | | | |
| | Before sterilization | After sterilization | Before sterilization | After sterilization |
| Characteristics | Almost | Almost | Light yellow | Light yellow |
| | colorless clear | colorless clear | clear liquid | clear liquid |
| | liquid | liquid | | |
| pH value | 2.02 | 2.03 | 2.51 | 2.51 |
| Chromium | 100.26% | 100.18% | 100.03% | 100.07% |
| Copper | 100.04% | 100.11% | 100.11% | 100.17% |
| Ferrum | 99.98% | 100.01% | 100.33% | 100.39% |
| Manganese | 100.18% | 100.15% | 100.36% | 100.20% |
| Iodine | 99.87% | 56.21% | 100.54% | 78.85% |
| Fluorine | 100.74% | 100.59% | 100.43% | 100.37% |
| Molybdenum | 100.52% | 100.48% | 100.60% | 100.55% |
| Selenium | 101.33% | 101.12% | 100.97% | 101.03% |
| Zinc | 100.03% | 100.15% | 100.12% | 100.08% |

The test results show that if the Addaven product does not contain xylitol as a stabilizer (i.e. Comparative Example 4), a clear solution can be prepared at a lower pH value, but its iodine content is greatly reduced (by about 40%) after the sterilization; in the case where xylitol is contained as a stabilizer, a clear product can be prepared (i.e., Comparative Example 5), but its iodine content is also greatly reduced (by about 20%). An excess (about 120%) of iodide feed is required in the commercial production of Addaven product.

A comparison between the examples of the present application and Comparative Examples 4, 5 shows that the formulation of the present application has a change of iodine content by no more than ±3% before and after the sterilization, indicating that the iodine element can be more stable even without using a stabilizer in the formulation of the present application.

Taking the sample of Example 2 as an example, the trace element composition injection of the present application was subjected to accelerated stability test, long-term stability test and compatibility stability test.

### 1) Accelerated Stability and Long-Term Stability Tests

Polypropylene ampoule inner packaging material, paper box outer packaging; accelerated stability test (temperature 40° C.±2° C., RH25%±5%) and long-term stability test (temperature 25° C.±2° C., RH60%±5%) were carried out respectively. The results are shown in Tables 15 and 16.

TABLE 15
Accelerated stability test results

| | | Time (month) | | |
|---|---|---|---|---|
| Item | | 0 | 3 | 6 |
| Characteristics | | Light yellow | Light yellow | Light yellow |
| | | clear liquid | clear liquid | clear liquid |
| pH value | | 2.92 | 2.93 | 2.92 |
| Visible foreign matter | | Meeting the | Meeting the | Meeting the |
| | | requirements | requirements | requirements |
| Insoluble | | Meeting the | Meeting the | Meeting the |
| microparticles | | requirements | requirements | requirements |
| Content (%) | Ferrum | 100.1 | 100.9 | 101.8 |
| | Zinc | 100.7 | 101.1 | 102.2 |
| | Copper | 100.5 | 101.3 | 102.0 |
| | Manganese | 100.8 | 101.7 | 102.4 |
| | Molybdenum | 100.4 | 101.6 | 102.2 |
| | Chromium | 100.3 | 101.1 | 101.8 |
| | Selenium | 100.6 | 101.4 | 101.9 |
| | Fluorine | 100.3 | 101.5 | 101.8 |
| | Iodine | 100.4 | 100.9 | 101.5 |

| | | | |
|---|---|---|---|
| Water loss rate (%) | 0 | 1 | 2 |

TABLE 16
Long-term stability test results

| | | Time (month) | | |
|---|---|---|---|---|
| Item | | 0 | 6 | 12 |
| Characteristics | | Light yellow | Light yellow | Light yellow |
| | | clear liquid | clear liquid | clear liquid |
| pH value | | 2.92 | 2.91 | 2.92 |
| Visible foreign matter | | Meeting the | Meeting the | Meeting the |
| | | requirements | requirements | requirements |
| Insoluble | | Meeting the | Meeting the | Meeting the |
| microparticles | | requirements | requirements | requirements |
| Content (%) | Ferrum | 100.1 | 100.2 | 100.3 |
| | Zinc | 100.7 | 100.1 | 100.4 |
| | Copper | 100.5 | 100.5 | 100.1 |
| | Manganese | 100.8 | 100.6 | 100.9 |
| | Molybdenum | 100.4 | 100.3 | 100.2 |
| | Chromium | 100.3 | 100.2 | 100.7 |
| | Selenium | 100.6 | 100.5 | 100.5 |
| | Fluorine | 100.3 | 100.5 | 100.4 |
| | Iodine | 100.4 | 100.3 | 100.1 |

| | | | |
|---|---|---|---|
| Water loss rate (%) | 0 | 0.1 | 0.2 |

The results show that all indexes of the accelerated stability test have no obvious change and meet the relevant requirements of the drug, and the contents are slightly increased due to slight water loss of the package under the conditions of high temperature and low humidity, but they all meet the requirements; under the long-term stability test conditions (temperature 25° C.±2° C., RH60%±5%), the water loss rate is less than 0.3% in 12 months, and all quality indexes have no obvious change.

### 2) Compatibility Stability Test

Compatibility stability tests with 0.9% sodium chloride injection, 5% glucose injection, 10% glucose injection, compound amino acid injection (20 AA), medium-long chain fat emulsion injection (C₆₋₂₄) and clinical “all in one” were carried out respectively. The compatibility test with sodium chloride injection, glucose injection, compound amino acid injection and medium-long chain fat emulsion injection was carried out as follows: one count (10 mL) of the injection was added into 500 ml of the compatibility solutions respectively, the solution, if less than 500 mL, being converted in proportion; as the “all in one” compatibility solution, commercial Kabiven, namely fat emulsion, amino acid (17) and glucose (11%) injection with specification of 1440 mL was used, to which were added one count (10 mL) of the product as well as water-soluble vitamin for injection and fat-soluble vitamin (II) for injection each in one count. The fat emulsion injection and the “all-in-one” compatibility system were complex, for which the characteristics, pH, osmotic pressure, particle size and particle size distribution as well as PFAT5 index were mainly detected.

Taking the 0-hour content as 100%, in the case of compatibility with glucose injection, reference should be made to the Pharmacopoeia of the People's Republic of China (2020) with regard to 5-hydroxymethylfurfural in glucose injection and the «Study on the Limit Inspection of 5-hydroxymethylfurfural in Vitamin C Glucose Injection» with regard to 5-hydroxymethylfurfural limit and method. The detection limit by high-performance liquid chromatography should not exceed 0.02%. The contents of some amino acids were detected by an amino acid analyzer. The test results are shown in Tables 17 to 22.

TABLE 17
Compatibility stability test results with 0.9% sodium chloride injection

| Item | | 0 hour | 8 hours | 12 hours | 24 hours |
|---|---|---|---|---|---|
| Characteristics | | Slightly | Slightly | Slightly | Slightly |
| | | yellow clear | yellow clear | yellow clear | yellow clear |
| | | liquid | liquid | liquid | liquid |
| pH value | | 4.18 | 4.19 | 4.19 | 4.20 |
| Osmotic pressure | | 273.4 | 275.0 | 278.3 | 279.0 |
| (mOsm/L) | | | | | |
| Visible foreign matter | | Meeting the | Meeting the | Meeting the | Meeting the |
| | | requirements | requirements | requirements | requirements |
| Insoluble | | Meeting the | Meeting the | Meeting the | Meeting the |
| microparticles | | requirements | requirements | requirements | requirements |
| Content (%) | Ferrum | 100.0 | 99.8 | 98.9 | 99.8 |
| | Zinc | 100.0 | 100.5 | 100.3 | 100.8 |
| | Copper | 100.0 | 99.7 | 99.9 | 100.4 |
| | Manganese | 100.0 | 100.2 | 100.5 | 100.6 |
| | Molybdenum | 100.0 | 99.9 | 100.2 | 99.6 |
| | Chromium | 100.0 | 100.3 | 100.6 | 100.2 |
| | Selenium | 100.0 | 100.6 | 99.9 | 99.7 |
| | Fluorine | 100.0 | 99.8 | 99.8 | 98.9 |
| | Iodine | 100.0 | 100.4 | 99.7 | 99.7 |

TABLE 18
Compatibility stability test results with 5% glucose injection

| Item | | 0 hour | 8 hours | 12 hours | 24 hours |
|---|---|---|---|---|---|
| Characteristics | | Slightly | Slightly | Slightly | Slightly |
| | | yellow clear | yellow clear | yellow clear | yellow clear |
| | | liquid | liquid | liquid | liquid |
| pH value | | 4.03 | 4.05 | 4.03 | 4.06 |
| Osmotic pressure | | 254.3 | 256.5 | 258.1 | 255.6 |
| (mOsm/L) | | | | | |
| Visible foreign matter | | Meeting the | Meeting the | Meeting the | Meeting the |
| | | requirements | requirements | requirements | requirements |
| Insoluble | | Meeting the | Meeting the | Meeting the | Meeting the |
| microparticles | | requirements | requirements | requirements | requirement |
| Content (%) | Ferrum | 100.0 | 99.9 | 100.2 | 99.9 |
| | Zinc | 100.0 | 100.2 | 100.1 | 100.3 |
| | Copper | 100.0 | 100.5 | 99.8 | 100.1 |
| | Manganese | 100.0 | 100.2 | 100.6 | 100.3 |
| | Molybdenum | 100.0 | 99.8 | 100.1 | 99.8 |
| | Chromium | 100.0 | 100.1 | 100.1 | 100.3 |
| | Selenium | 100.0 | 100.7 | 100.6 | 99.8 |
| | Fluorine | 100.0 | 100.2 | 99.9 | 100.5 |
| | Iodine | 100.0 | 100.2 | 99.6 | 99.3 |

| | | | | |
|---|---|---|---|---|
| 5-hydroxymethylfurfural | 0.00541 | 0.00540 | 0.00539 | 0.00541 |
| (%) | | | | |

TABLE 19
Compatibility stability test results with 10% glucose injection

| Item | | 0 hour | 8 hours | 12 hours | 24 hours |
|---|---|---|---|---|---|
| Characteristics | | Slightly | Slightly | Slightly | Slightly |
| | | yellow clear | yellow clear | yellow clear | yellow clear |
| | | liquid | liquid | liquid | liquid |
| pH value | | 3.73 | 3.75 | 3.74 | 3.75 |
| Osmotic pressure | | 484.2 | 485.1 | 486.4 | 485.4 |
| (mOsm/L) | | | | | |
| Visible foreign matter | | Meeting the | Meeting the | Meeting the | Meeting the |
| | | requirements | requirements | requirements | requirements |
| Insoluble | | Meeting the | Meeting the | Meeting the | Meeting the |
| microparticles | | requirements | requirements | requirements | requirements |
| Content (%) | Ferrum | 100.0 | 100.1 | 100.3 | 99.8 |
| | Zinc | 100.0 | 100.3 | 100.3 | 100.5 |
| | Copper | 100.0 | 100.2 | 99.7 | 99.8 |
| | Manganese | 100.0 | 100.1 | 99.6 | 99.9 |
| | Molybdenum | 100.0 | 99.9 | 99.7 | 100.3 |
| | Chromium | 100.0 | 100.2 | 100.4 | 100.5 |
| | Selenium | 100.0 | 99.7 | 99.5 | 99.7 |
| | Fluorine | 100.0 | 99.7 | 99.8 | 100.1 |
| | Iodine | 100.0 | 99.8 | 99.6 | 99.5 |

| | | | | |
|---|---|---|---|---|
| 5-hydroxymethylfurfural | 0.00464 | 0.00463 | 0.00464 | 0.00463 |
| (%) | | | | |

TABLE 20
Compatibility stability test results with compound amino acid injection
(20 AA)

| Item | | 0 hour | 8 hours | 12 hours | 24 hours |
|---|---|---|---|---|---|
| Characteristics | | Slightly | Slightly | Slightly | Slightly |
| | | yellow clear | yellow clear | yellow clear | yellow clear |
| | | liquid | liquid | liquid | liquid |
| pH value | | 6.23 | 6.25 | 6.24 | 6.25 |
| Osmotic pressure | | 861.2 | 864.3 | 867.2 | 865.5 |
| (mOsm/L) | | | | | |
| Visible foreign matter | | Meeting the | Meeting the | Meeting the | Meeting the |
| | | requirements | requirements | requirements | requirements |
| Insoluble | | Meeting the | Meeting the | Meeting the | Meeting the |
| microparticles | | requirements | requirements | requirements | requirements |
| Content (%) | Ferrum | 100.0 | 100.1 | 100.3 | 99.8 |
| | Zinc | 100.0 | 100.3 | 100.3 | 100.5 |
| | Copper | 100.0 | 100.2 | 99.7 | 99.8 |
| | Manganese | 100.0 | 100.1 | 99.6 | 99.9 |
| | Molybdenum | 100.0 | 99.9 | 99.7 | 100.3 |
| | Chromium | 100.0 | 100.2 | 100.4 | 100.5 |
| | Selenium | 100.0 | 99.7 | 99.5 | 99.7 |
| | Fluorine | 100.0 | 99.7 | 99.8 | 100.1 |
| | Iodine | 100.0 | 99.8 | 99.6 | 99.5 |

| | | | | |
|---|---|---|---|---|
| Methionine (%) | 100.0 | 100.2 | 100.1 | 99.7 |
| Acetyl tyrosine (%) | 100.0 | 99.4 | 100.2 | 100.3 |
| Aspartic acid (%) | 100.0 | 100.5 | 100.2 | 100.4 |
| Threonine (%) | 100.0 | 100.2 | 100.0 | 100.1 |
| Serine (%) | 100.0 | 100.3 | 100.4 | 100.6 |
| Glutamic acid (%) | 100.0 | 99.7 | 99.6 | 99.8 |
| Glycine (%) | 100.0 | 100.5 | 99.8 | 99.7 |
| Alanine (%) | 100.0 | 100.4 | 100.1 | 100.2 |
| Valine (%) | 100.0 | 100.2 | 100.8 | 100.3 |
| Leucine (%) | 100.0 | 100.5 | 100.2 | 100.3 |
| Isoleucine (%) | 100.0 | 99.8 | 99.7 | 99.4 |
| Phenylalanine (%) | 100.0 | 100.3 | 100.5 | 100.2 |
| Histidine (%) | 100.0 | 100.4 | 100.7 | 100.6 |
| Tryptophan (%) | 100.0 | 100.0 | 99.7 | 98.9 |
| Ornithine | 100.0 | 99.6 | 99.4 | 99.3 |
| hydrochloride (%) | | | | |
| Lysine acetate (%) | 100.0 | 99.4 | 100.1 | 100.2 |
| Proline (%) | 100.0 | 100.3 | 100.0 | 99.5 |

TABLE 21
Compatibility stability test results with medium-long chain fat
emulsion injection (C₆₋₂₄)

| Item | | 0 hour | 12 hours | 24 hours |
|---|---|---|---|---|
| Characteristics | | Milky white | Milky white | Milky white |
| | | liquid | liquid | liquid |
| pH value | | 7.67 | 7.68 | 7.68 |
| Osmotic pressure (mOsm/L) | | 386.2 | 387.3 | 385.1 |
| Particle | Average particle | 256.5 | 257.4 | 257.3 |
| size and | diameter (nm) | | | |
| particle | PDI | 0.116 | 0.125 | 0.137 |
| size distribution | (polydispersity | | | |
| | index) | | | |

| | | | |
|---|---|---|---|
| PFAT5 | 0.003% | 0.005% | 0.008% |

TABLE 22
Compatibility stability test results with Kabiven “all in one”

| Item | | 0 hour | 12 hours | 24 hours |
|---|---|---|---|---|
| Characteristics | | Milky | Milky | Milky |
| | | white | white | white |
| | | liquid | liquid | liquid |
| pH value | | 5.51 | 5.52 | 5.54 |
| Osmotic pressure (mOsm/L) | | 760.5 | 758.7 | 762.6 |
| Particle size | Average particle | 332.2 | 335.3 | 345.6 |
| and particle | diameter (nm) | | | |
| size distribution | PDI (polydis- | 0.107 | 0.105 | 0.108 |
| | persity index) | | | |

| | | | |
|---|---|---|---|
| PFAT5 | 0.01% | 0.01% | 0.01% |

As can be seen from the above compatibility stability tests, the composition injection of the present application has good compatibility stability with common compatibility solutions.

### 3) Safety Test

Sample (test sample group) of Example 2 and Addaven product (control sample group, Lot No.: 12NHB19, source: Fresenius Kabi Limited) were taken for comparison of safety tests, including systemic active allergic reaction test in guinea pigs, hemolysis test in New Zealand male rabbit, and vascular irritation test in New Zealand male rabbit.

Systemic active allergic reaction test in guinea pigs: Hartley guinea pigs, SPF grade, source: Beijing Vital River Laboratory Animal Technology Co., Ltd., animal production license No.: SCXK (JING) 2016-0011, test animal certification No.: No. 110011201110221326, issuer: Beijing Science and Technology Commission, age at the start of administration: 5-6 weeks.

Guinea pigs were sensitized by intraperitoneal injection of the sample of Example 2 and Addaven every other day for three consecutive days. In the allergic reaction test, 0.9% sodium chloride injection (Lot No.: 6B20060706, Shandong Qidu Pharmaceutical Co., Ltd.) was selected as the compatibility solution. Test sample low-dose group was administered with the clinically intended concentration of the test sample solution, and test sample high-dose group was administered with twice the concentration of the test sample solution. The administration volume was 0.5 mL/animal for each group. On the 14^{th} and 21^{st} days after the last sensitization, twice the sensitization dose of the product was intravenously injected for irritation, respectively.

Test sample low-dose group: 4 mL of the sample of Example 2 was added to 40 mL of 0.9% sodium chloride injection in a clean bench, and shaken well to obtain an administration preparation for the test sample low-dose group, which was stored at normal temperature in dark in a sealed manner.

Test sample high-dose group: 5 mL of the sample of Example 2 was added to 25 mL of 0.9% sodium chloride injection in a clean bench, and shaken well to obtain an administration preparation for the test sample high-dose group, which was stored at normal temperature in dark in a sealed manner.

Control sample group: using Addaven product, an administration preparation was prepared and stored according to the same method as the test sample high-dose group.

Negative control group: 0.9% sodium chloride injection;

Positive control group: a proper amount of bovine serum albumin (Lot No. 128K054, Beijing Solebao Science and Technology Co., Ltd.) was weighed and prepared into a bovine serum albumin solution with a concentration of 40 mg/ml using 0.9% sodium chloride injection.

The systemic active allergic reaction test design in guinea pigs is shown in Table 23.

TABLE 23
Administration volume Administration dose Number

| | | | | | | Number |
|---|---|---|---|---|---|---|
| | | Administration | | Administration | | of animals |
| | | volume | | dose | | (head) |
| | | (mL/each) | | (mg/each) | | female/ |
| Group | Concentration | Sensitization | Excitation | Sensitization | Excitation | male |
| Negative | / | 0.5 | 1.0 | / | / | 3/3 |
| control | | | | | | |
| group | | | | | | |
| Test sample | / | 0.5 | 1.0 | / | / | 3/3 |
| low-dose | | | | | | |
| group | | | | | | |
| Test sample | / | 0.5 | 1.0 | / | / | 3/3 |
| high-dose | | | | | | |
| group | | | | | | |
| Control | / | 0.5 | 1.0 | / | / | 3/3 |
| sample | | | | | | |
| group | | | | | | |
| Positive | 40 mg/mL | 0.5 | 1.0 | 20 | 40 | 3/3 |
| control | | | | | | |
| group | | | | | | |

Test results: no allergic reactions are observed in the animals in each dose group of the sample of Example 2 and Addaven, which is consistent with the result of the negative control group (0.9% sodium chloride injection). During the test, no abnormal reactions are observed in the clinical observation of the relevant animals, and the weight gain of the animals in each group is normal. After irritation on the 14^{th} day after the last sensitization, the animals in the positive control group show symptoms of allergic reactions, including standing hair, scratching the nose, coughing, urination, difficulty breathing, wheezing, unstable gait, spasms, tidal breathing, and death, the allergic reactions being strongly positive to extremely strong positive; after irritation on the 21^{st} day after the last sensitization, they show symptoms of allergic reactions, including standing hair, scratching the nose, sneezing, coughing, urination, defecation, difficulty breathing, wheezing, unstable gait, spasms, tidal breathing, and death, the allergic reactions being strongly positive to extremely strong positive.

The systemic active allergic reaction test in guinea pigs shows that the allergic reactions with the injection of the present application and Addaven are both negative.

### 4) In Vitro Hemolysis Test on Rabbit Red Blood Cells

2% rabbit red blood cell suspension: 8 mL of blood was collected from a central ear artery of one healthy New Zealand male rabbit, placed in an Erlenmeyer flask filled with glass beads, and shaken for about 10 minutes. Then, the blood was stirred with a glass rod, and fibrinogen was removed to obtain defibrillated blood. Red blood cells were aspirated using a pipette, to which was then added 10 times the volume of sodium chloride injection, shaken well, and centrifuged at 1500 rpm for 10 minutes, the supernatant was removed, and the precipitated red blood cells were then washed with sodium chloride injection 4 times in the same manner, for a total of 5 times, until the supernatant did not show any red color. 0.6 mL of the obtained red blood cells were taken, and prepared into a 2% suspension by volume using sodium chloride injection, for later use.

Test animal producer: Suzhou Genesc Biotechnology Co., Ltd., test animal quality certification No.: 20201103271, test animal production license No.: SCXK (SU) 2020-0002, issuer: Jiangsu Provincial Department of Science and Technology.

Test sample solution: 9 mL of the sample of Example 2 was added to 90 ml of 0.9% sodium chloride injection in a clean bench, and shaken well to obtain a test sample solution, which was stored at normal temperature in dark in a sealed manner.

Control sample solution: 9 mL of Addaven product was added to 90 ml of 0.9% sodium chloride injection in a clean bench, and shaken well to obtain a control sample solution, which was stored at normal temperature in dark in a sealed manner.

Negative control solution: 0.9% sodium chloride injection.

Positive control solution: sterilized water for injection (Lot No. 2A20032101, Shandong Qidu Pharmaceutical Co., Ltd.).

Clean test tubes were numbered as follows: the tubes Nos. 1-5 and Nos. 8-12 were test sample/control sample solution tubes respectively, the tubes Nos. 6 and 13 were negative control tubes (0.9% sodium chloride injection), and the tubes Nos. 7 and 14 were positive control tubes (sterilized water for injection). The solutions were added according to the following table, shaken gently and then immediately incubated in a 37.0° C. incubator. Observation was made 1 time every 15 minutes starting from the placement of the solution in the incubator (0 h before incubation), then 1 time every 1 hour after 1 hour, for a total of 3 hours, and each observation result was recorded.

TABLE 24
In vitro hemolysis test design table on rabbit red blood cells

| Test tube No. | 1, 8 | 2, 9 | 3, 10 | 4, 11 | 5, 12 | 6, 13 | 7, 14 |
|---|---|---|---|---|---|---|---|
| 2% red blood cell | 2.5 | 2.5 | 2.5 | 2.5 | 2.5 | 2.5 | 2.5 |
| suspension (mL) | | | | | | | |
| 0.9% sodium chloride | 2.4 | 2.3 | 2.2 | 2.1 | 2.0 | 2.5 | — |
| injection (mL) | | | | | | | |
| Sterilized water for | — | — | — | — | — | — | 2.5 |
| injection (mL) | | | | | | | |
| Test sample solution | 0.1 | 0.2 | 0.3 | 0.4 | 0.5 | — | — |
| (mL) | | | | | | | |

Test results: during the test, after incubation of the 0.9% sodium chloride injection tubes (Nos. 6 and 13) at 37.0° C. for 3 hours, a large amount of red blood cells sink to the bottom of the tube, the supernatant is colorless and clear, and the sunk red blood cells are redispersed in each tube after shaking well, without hemolysis and coagulation; after incubation of the sterilized water for injection tubes (Nos. 7 and 14) at 37.0° C. for 3 hours, the solution presents clear red with no residual red blood cells at the bottom of the tube, indicating complete hemolysis. In the case of incubating the tubes Nos. 1-5 and Nos. 8-12, which are test sample/control sample solution tubes respectively, at 37.0° C., starting from the 30^{th} minute, the red blood cell sinking rate of each of the test sample/control sample solution tubes is faster than that of the negative control tube, and, after incubation for 3 hours, each supernatant is colorless and clear, without hemolysis. After shaking well, the sunk red blood cells cannot be evenly dispersed, and all of them exhibit a slight coagulation phenomenon.

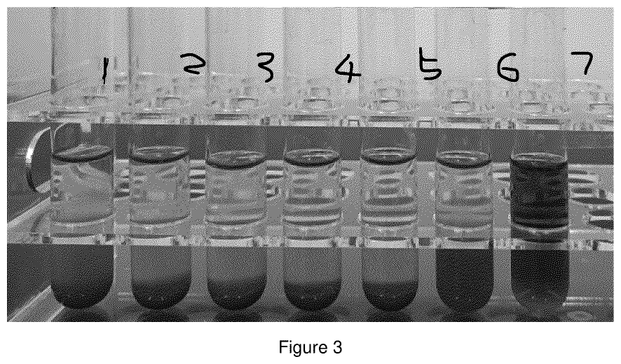

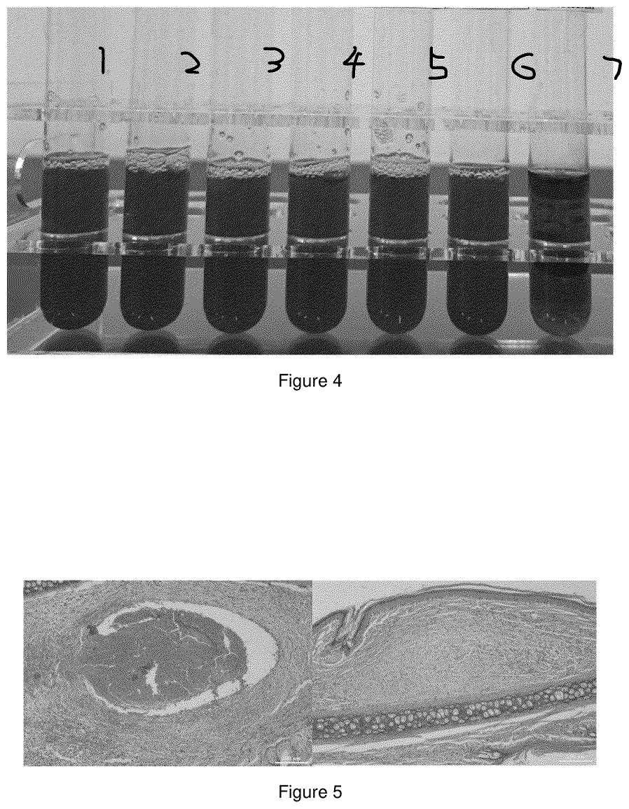

The hemolysis test results show that the multi-trace element composition injection of the present application is consistent with Addaven product, both of which have no in vitro hemolysis effect on rabbit red blood cells, and have slight coagulation effect.

Wherein, FIG. 2 is a photograph of the tubes Nos. 1-7 (the tubes are numbered 1, 2, 3, 4, 5, 6 and 7 in sequence from left to right) shaken well after sample addition; FIG. 3 is a photograph of the tubes Nos. 1-7 3 hours after sample addition; FIG. 4 is a photograph of the tubes Nos. 1-7 shaken well 3 hours after sample addition.

### 4) Vascular Irritation Test

10 New Zealand rabbits, 3-4 months old, clean grade, half male and half female, supplier: Qingdao Kangda Biotechnology Co., Ltd., license No.: SCXK (LU) 20210003, test animal quality certification No.: 370823211100104051.

The preparation methods of test sample solution, control sample solution and negative control solution are the same as those under the item of in vitro hemolysis test.

In the test, 10 mL of the sample of Example 2+100 mL of 0.9% sodium chloride injection (test sample group), 10 mL of Addaven product+100 ml of 0.9% sodium chloride injection (control sample group) were respectively administered according to 5.2 mL/kg, and meanwhile, 0.9% sodium chloride injection was used as the negative control, the administration volume being the same as that of the test sample. The dosage design and grouping are shown in Table 25.

TABLE 25
Vascular irritation test design table

| | Administration | Administration | Dissection time | |
|---|---|---|---|---|
| | volume | speed | End of | End of |
| Group | mL/kg | mL/h | administration | recovery |
| Test sample | 5.2 | 30 | T1~T3 | T4, T5 |
| group | | | | |
| Control | 5.2 | 30 | R1~R3 | R4, R5 |
| sample | | | | |
| group | | | | |
| Negative | 5.2 | 30 | — | — |
| control | | | | |
| group | | | | |

Administration route: marginal ear vein drip administration, administration frequency and duration: administered 1 time per day for consecutive 7 days.

Recovery period: the animals were observed for 72 hours after the last administration, histopathological examination was performed on some animals through autopsy, and the observation continued for 14 days in the recovery period.

Administration method: each group of New Zealand rabbits was administered using the same body left and right self-contrast method. In this test, a microinjection pump was used for intravenous drip administration. The left ear was administered with the test sample solution or the control sample solution, and the right ear was administered with 0.9% sodium chloride injection as a control using the same method.

Detailed clinical observation: observed 1 time per day. Observation contents: including but not limited to physical signs, general activity, mental status, respiratory status, fecal characteristics, death, and the like.

Body weight test time and animals: all animals on the day of first administration, and animals to be dissected on the day of sacrifice and sampling were weighed 1 time each.

Local irritation reaction at the injection site was visually observed 1 time per day before administration on administration days, and on non-administration days. 72 hours after the last administration and 14 days after continued recovery, corresponding animals were euthanized under anesthesia (numbers being shown in the table above, T representing the test sample group, R representing the control sample group). Both ears of the rabbit were cut at the ear root, and the specimens were fixed in 10% neutral formalin solution. Upon sampling, dehydration, paraffin embedding, sectioning, and HE staining, then histopathological examination was performed.

Results: during the test, the general clinical signs of New Zealand rabbits in each group are good, and no obvious abnormalities are observed. The body weight of the surviving New Zealand rabbits in each group fluctuates within the normal range, and no obvious abnormal changes are observed.

In the period from the 5^{th} day to the 10^{th} day, the animals in the control sample group all show intravascular congestion at the administration site on the administration side, whereas no obvious abnormalities are visually observed at the administration site in both the test sample group of the present application, and the above animals on the control side.

72 hours after the last administration, the blood vessels at the injection site, and the proximal non-injection site on the administration side of all animals in the control sample group are purple red. Microscopic examination shows that intravascular congestion, vasodilation and infiltration of inflammatory cells in the perivascular tissue are observed at the proximal non-injection site of the ear vein on the administration side of all animals in the control sample group, suggesting that the marginal ear vein drip administration of the control sample can lead to obvious irritation of the ear vein and surrounding tissues of New Zealand rabbits. There are no abnormalities in the blood vessels at the injection site of the test sample group and other animals on the control side.

At the end of the recovery period, all animals in the test sample group and the control sample group show no abnormalities at the distal non-injection site, injection site, and proximal non-injection site on the administration side and the control side. The vascular patterns are clear, without expansion, congestion, and no pathological changes such as bleeding and edema are observed in the perivascular tissues.

The vascular irritation test show that, 72 hours after the last administration, the control sample group has significant irritation to the ear vein and surrounding tissues of New Zealand rabbits with marginal ear vein drip administration 1 time per day for 7 consecutive days. In contrast, the test sample group of the present application has no obvious irritation. At the end of the recovery period, no vascular irritation reaction related to the test sample group and the control sample group is observed. Meanwhile, this indicates that the rabbits can be recovered after the recovery period with respect to the irritation in the control sample group.

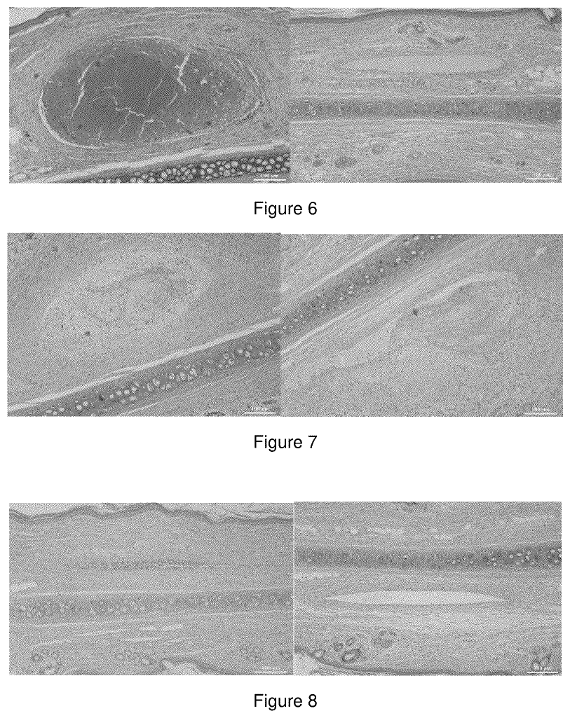

FIGS. 5 to 8 show typical diagrams of histopathological examination results of the animals with the control sample on the administration side and the control side, and with the test sample injection of the patented composition of the present application on the administration side and the control side. At the end of the recovery period, no abnormality is found in the control groups, so no diagram is shown therefor in the figures.

A sample of Comparative Example 1 (Lot No.: 20220501, source: Beijing Zang WeiXinkang Pharmaceutical R & D Co., Ltd.) was subjected to vascular irritation test in the same manner as described above.

### 5 New Zealand Rabbits, 3-4 Months Old, Clean Grade, 2 Female and 3 Male.

Test sample group: 10 mL of the sample of Comparative Example 1 was added to 100 mL of 0.9% sodium chloride injection in a clean bench, and shaken well to obtain a test sample solution, which was stored at normal temperature in dark in a sealed manner.

Negative control group: 0.9% sodium chloride injection.

The test sample and the negative control were respectively administered according to 5.2 mL/kg. The dosage design and grouping are shown in Table 26.

TABLE 26
Vascular irritation test design table

| | Administration | Administration | Dissection time | |
|---|---|---|---|---|
| | volume | speed | End of | End of |
| Group | mL/kg | mL/h | administration | recovery |
| Test sample | 5.2 | 30 | T1~T3 | T4, T5 |
| group | | | | |
| Negative | 5.2 | 30 | — | — |
| control | | | | |
| group | | | | |

Administration route: marginal ear vein drip administration, administration frequency and duration: administered 1 time per day for consecutive 7 days.

Recovery period: the animals were observed for 72 hours after the last administration, histopathological examination was performed on some animals through autopsy, and the observation continued for 14 days in the recovery period.

Administration method: each group of New Zealand rabbits was administered using the same body left and right self-contrast method. In this test, a microinjection pump was used for intravenous drip administration. The left ear was administered with the test sample solution, and the right ear was administered with 0.9% sodium chloride injection as a negative control using the same method.

Detailed clinical observation: observed 1 time per day. Observation contents: including but not limited to physical signs, general activity, mental status, respiratory status, fecal characteristics, death, and the like.

Body weight test time and animals: all animals on the day of first administration, and animals to be dissected on the day of sacrifice and sampling were weighed 1 time each.

Local irritation reaction at the injection site was visually observed 1 time per day before administration on administration days, and on non-administration days. 72 hours after the last administration and 14 days after continued recovery, corresponding animals were euthanized under anesthesia (numbers being shown in the table above, T representing the test sample group). Both ears of the rabbit were cut at the ear root, and the specimens were fixed in 10% neutral formalin solution. Upon sampling, dehydration, paraffin embedding, sectioning, and HE staining, histopathological examination was performed.

Results: during the test, one male New Zealand rabbit dies on the 3^{rd} day after the last administration, with mild convulsions in the limbs, perianal contamination, and prone position. This animal has a small to moderate amount of brown loose/soft feces from the 3^{rd} day after the initial administration to the 2^{nd} day after the last administration. After death, the body weight decreases by 36% compared to that before administration, no obvious abnormal change is observed in gross anatomy, and no lethal lesion is observed under a microscope. Apart from the dead animal, another male animal shows a small amount of soft feces on the 3^{rd} day after the initial administration, a large amount of brown loose feces on the 2^{nd} to 4^{th} days after the last administration, and a small amount of brown soft feces afterwards; a female animal shows a small amount of brown loose feces from the 4^{th} day after the initial administration to the 3^{rd} day after the last administration, while the other 2 animals show no obvious abnormal reaction in the general clinical observation.

During the whole administration and recovery period, no obvious irritation abnormality is visually observed in the animals including the dead one at the administration site on the test sample side and the control side. The animals show no abnormalities at the distal non-injection site, injection site, and proximal non-injection site on the administration side and the control side, and no pathological changes such as bleeding and edema are observed in the perivascular tissues.

Although no significant abnormality is observed in the vascular irritation of the animals administered with the sample of Comparative Example 1 in which the zinc dose is significantly increased, death of animals and gastrointestinal side effects in some animals are observed with the formulation of Comparative Example 1 containing a higher zinc dose.

### Comparative Example 6

The dose of zinc gluconate was increased to 153 μmol.

TABLE 27
Formulation (metered in 10 mL)

| | | Content of |
|---|---|---|
| Composition | Effective element | effective element |
| Ferrous gluconate | Ferrum | 18 μmol |
| Zinc gluconate | Zinc | 153 μmol |
| Copper gluconate | Copper | 4.7 μmol |
| Manganese gluconate | Manganese | 1 μmol |
| Sodium fluoride | Fluorine | 50 μmol |
| Potassium iodide | Iodine | 1 μmol |
| Sodium selenite | Selenium | 0.9 μmol |
| Sodium molybdate | Molybdenum | 0.2 μmol |
| Chromium chloride | Chromium | 0.2 μmol |
| Hydrochloric acid | pH regulator | Proper amount |

According to the above formulation, 350 L of product was prepared in view of a theoretical quantity of 35,000 counts, the operation being carried out in a dark environment.

Water for injection was added to a liquid preparation tank, while starting and keeping it in a stirring state, ferrous gluconate, zinc gluconate, copper gluconate, manganese gluconate, sodium fluoride were dissolved or suspended in a plastic beaker with a proper amount of water for injection respectively, the resulting solutions or suspensions were added to the liquid preparation tank, followed by dissolution with stirring, the plastic beaker was rinsed with a proper amount of water for injection, and the rinse water was merged into the liquid preparation tank; sodium selenite, sodium molybdate, chromium chloride and potassium iodide were dissolved in plastic beakers with a proper amount of water for injection respectively, the resulting solutions were added to the liquid preparation tank, the plastic beakers were rinsed with water for injection, the rinse water was merged into the liquid preparation tank, a 1M hydrochloric acid solution was added to adjust the pH value to be 2.8~3.0, water for injection was added to full volume, and the resulting liquid medicine was filtered by a filter containing two 0.22 μm filter cartridges in series to a buffer tank before a potting machine, ready for potting.

A BFS plastic ampoule potting machine conveyed polypropylene resin to a hopper of a BFS extruder through a vacuum material suction device, the liquid medicine in the buffer tank was pressed into a liquid separation device according to a specified program, ampoule manufacture and potting were started, with a loading amount of 10 ml/count, the potted product was conveyed to a sterilization procedure through a conveying belt, and sterilized at the temperature of 121° C. for 12 minutes.

The formulation process of Example 2 and the sample of Comparative Example 6 were detected. The element contents were expressed taking the amount of the formulation as 100%. The contents of formic acid and oxalic acid, which were impurities from degradation of gluconic acid, were detected by ion chromatography, and the percentages were calculated in terms of the theoretical dose of gluconic acid in the formulation. The results are shown in Table 28.

TABLE 28
Comparison of detection results before and after the sterilization in
Example 2 and Comparative Example 6

| | Detection index | | | |
|---|---|---|---|---|
| | Formulation of Example 2 | | Comparative Example 6 | |
| | Before | After | Before | After |
| | sterilization | sterilization | sterilization | sterilization |
| Characteristics | Light yellow | Light yellow | Light yellow | Light yellow |
| | clear liquid | clear liquid | clear liquid | clear liquid |
| | | | | (darker) |
| pH value | 2.9 | 2.9 | 2.9 | 3.0 |
| Ferrum | 100.2% | 100.1 | 100.5% | 100.2% |
| Zinc | 100.4% | 100.7 | 100.3% | 100.4% |
| Copper | 99.9% | 100.5 | 100.2% | 101.1% |
| Manganese | 100.1% | 100.8 | 100.6% | 99.9% |
| Molybdenum | 100.1% | 100.4 | 100.0% | 100.8% |
| Chromium | 100.4% | 100.3 | 100.3% | 100.2% |
| Selenium | 100.5% | 100.6 | 100.7% | 101.2% |
| Fluorine | 101.1% | 100.3 | 100.9% | 100.1% |
| Iodine | 100.5% | 100.4 | 100.1% | 100.2% |
| Formic acid | 0.2% | 0.5% | 0.3% | 1.3% |
| Oxalic acid | 0.3% | 0.6% | 0.3% | 1.4% |

It can be seen from the results that, in the formulation of Example 2 and the formulation of Comparative Example 6, the pH and the contents have no obvious changes before and after the sterilization, and the clarity and color of the solution after the sterilization are darker than those before the sterilization, with more changes in the formulation of Comparative Example 6 after the sterilization versus before the sterilization compared to the formulation of Example 2; with regard to impurities from degradation, there are increases in their contents in both the formulation of Example 2 and the formulation of Comparative Example 6 after the sterilization than those before the sterilization, but the increases in the formulation of Comparative Example 6 are more significant than those in the formulation of Example 2. As calculated with the content of gluconic acid in the formulation of Example 2 being 40 mg and the content of gluconic acid in the formulation of Comparative Example 6 being 69 mg, the contents of formic acid and oxalic acid in each finished product with the formulation of Example 2 after the sterilization are 0.2 mg and 0.24 mg respectively, while the contents of formic acid and oxalic acid in each finished product with the formulation of Comparative Example 6 after the sterilization are 0.9 mg and 1 mg respectively, which are obviously higher than those of Example 2, indicating that the composition of the present application has lower impurity contents and further reduces the possible impurity safety risk.

### Plasma Element Concentration, White Blood Cell Number (WBC) and Repeated Administration Irritation Study

Trace element plasma concentration, white blood cell number (WBC) and administration irritation were evaluated by observing foreign body implantation malnutrition model rats after continuous intravenous injection of three formulations of trace element injections.

The test was performed using 40 male SD rats, grade: SPF grade, breeder: Beijing Vital River Laboratory Animal Technology Co., Ltd., production license No. and issuer: SCXK (JING) 2021-0011 (Beijing Science and Technology Commission).

The animals were fed with a feed (containing no trace elements) provided by Tianjin Keao Xieli Feed Co., Ltd. for more than 5 days, and subcutaneously implanted on the back with cotton balls and filter paper foreign body (keeping 8 animals as normal controls). On the 3^{rd} day after the operation, the animals were randomly divided into model control group, formulation 1 group (Addaven product), formulation 2 group (Comparative Example 6), and formulation 3 group (Example 2), 8 animals in each group. The test groups and the administration design are shown in Table 29.

TABLE 29
Test groups and administration design

| | | Administration | |
|---|---|---|---|
| | | volume | |
| Group | Test sample name | (ml/kg) | Animal No. |
| Normal control group | 0.9% sodium | 10 | M001~M008 |
| (Blank) | chloride injection | | |
| Model control group | 0.9% sodium | 10 | M009~M016 |
| (Model) | chloride injection | | |
| Formulation 1 group | Addaven | 10 | M017~M024 |
| (P1) | | | |
| Formulation 2 group | Comparative | 10 | M025~M032 |
| (P2) | Example 6 | | |
| Formulation 3 group | Example 2 | 10 | M033~M040 |
| (P3) | | | |

Starting from the 4^{th} day after the operation, the animals were subjected to tail vein administration 1 time per day for consecutive 7 days, with the administration volume of 10 ml/kg (in terms of formulations 1-3, the recommended clinical dose for adults is 10 mL per day; for 60 kg of human, the body surface area is 1.62686 m², for 200 g of rat, the body surface area is 0.03036 m², then the equivalent clinical dose for rats is about 0.93 mL/kg/d; in this test, the dose was designed to be 2.5 mL/kg, the injection was used after dilution by 4 times, and the administration volume was 10 mL/kg, which is about 2.7 times of the equivalent clinical dose for rats). The normal control group and the model control group were given 0.9% sodium chloride injection. Each formulation group was given the injection with the corresponding No.

PK blood collection: on the morning 1 day before the operation, the morning 1 day after the operation, 0 minute before the first administration, 2 minutes, 15 minutes, 1 hour, 4 hours after the first administration, 15 minutes after the 4^{th} day's administration, 15 minutes after the 7^{th} day's administration and the morning of the 10^{th} day, PK blood samples were collected by taking about 0.2 mL of venous blood from the jugular sinus of rats, the whole blood was put into an EDTA anticoagulant tube and centrifuged at 6000 rpm for 10 minutes. The blood plasma was temporarily stored in a refrigerator at −20° C. during the blood collection. At the end of the blood collection, the blood plasma was transferred to −70° C. for freezing storage, and then transferred to a central laboratory for detecting the contents of copper, manganese, zinc and selenium in the blood plasma by Agilen ICP-Ms method.

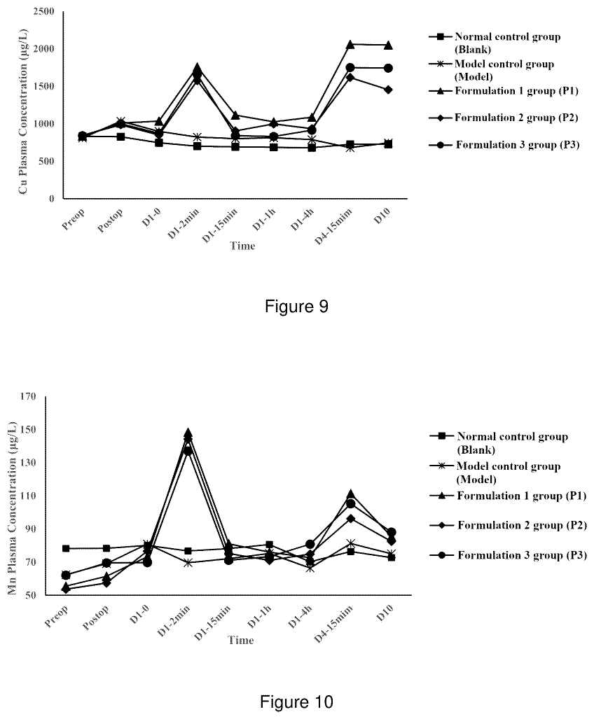

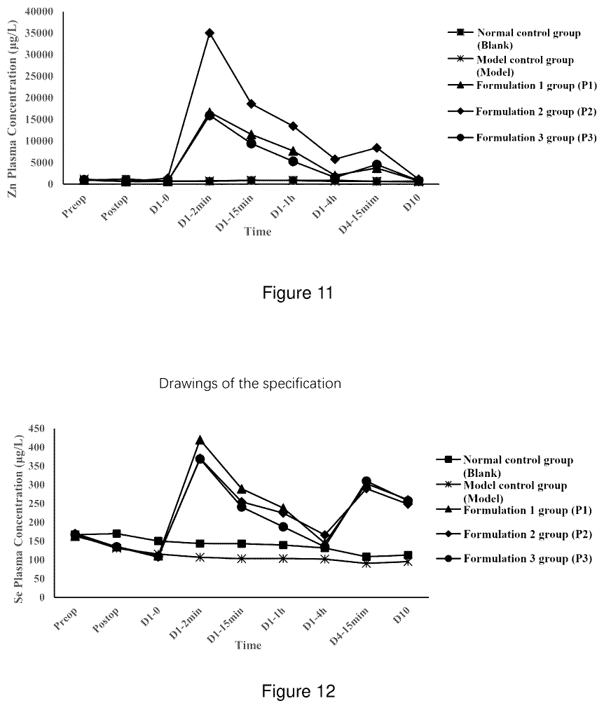

The ICP-Ms detection results of copper, manganese, zinc and selenium plasma concentrations show that the copper plasma concentration is slightly increased after the operation, the selenium plasma concentration is reduced, and the manganese and zinc concentrations have no obvious changes before and after the operation; the plasma concentrations are each significantly increased after the administration, each reaching a peak value after 2 minutes, the zinc concentrations in the formulation 2 group and the formulation 3 group are basically reduced to the level of the normal control group after 4 hours, the zinc concentration in the formulation 1 group is significantly higher than those in the formulation 2 group and the formulation 3 group, the detection results on the 10^{th} day after 7 days of consecutive administration show that the zinc plasma concentrations in the formulation 1 group, the formulation 2 group and the formulation 3 group are basically maintained at the normal level and are the same as that in the normal control group, the manganese plasma concentrations in the formulation 1 group, the formulation 2 group and the formulation 3 group are slightly higher than that in the normal control group, the copper and selenium plasma concentrations in the formulation 1 group, the formulation 2 group and the formulation 3 group are all maintained at higher levels and are significantly higher than those in the normal control group and the model control group. The plasma concentration results of copper, manganese, zinc and selenium are shown in FIG. 9, FIG. 10, FIG. 11 and FIG. 12.

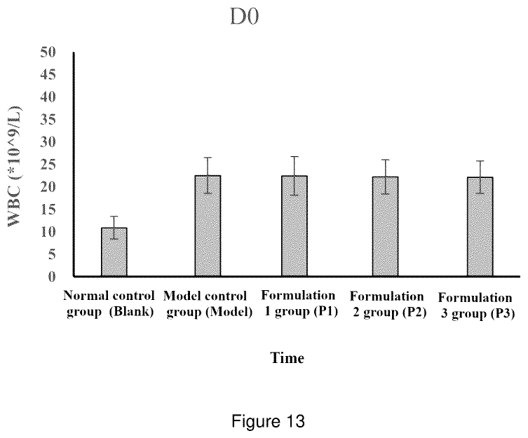

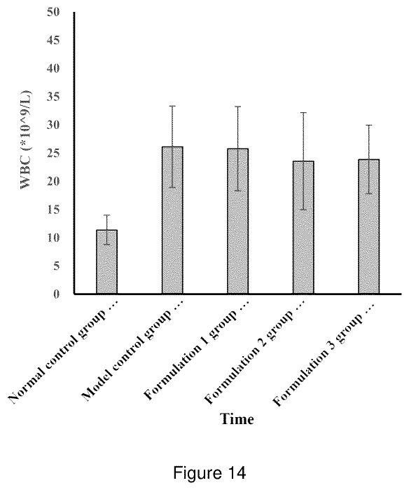

White blood cell number (WBC): blood samples were collected from the jugular sinus of rats on day 0 before the administration and the 4^{th} day of the administration, subjected to EDTA anticoagulation treatment, and detected using an XN-1000V (BI) full-automatic modular animal blood and body fluid analyzer (flow cytometry+semiconductor laser+nucleic acid fluorescence staining method). The results are shown in FIGS. 13 and 14.

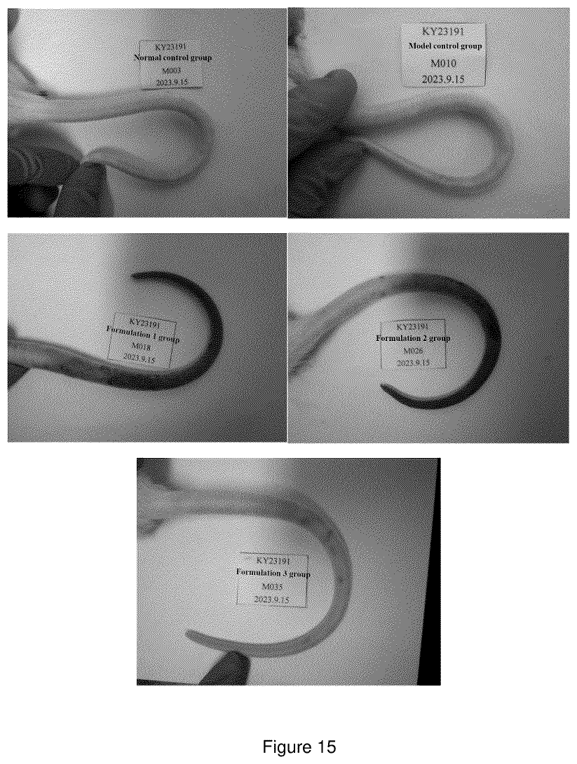

The results show that the white blood cell number is significantly increased in both the model group and the administration group after the operation. After 4 days of administration, the white blood cell number in each administration group is somewhat reduced compared to the model group, wherein the formulation 2 and the formulation 3 are comparable and better than the formulation 1. Typical diagrams of irritation after 4 consecutive days of tail vein administration in the normal control group, the model control group, the formulation 1 group, the formulation 2 group, and the formulation 3 group are shown in FIG. 15. Multiple tail vein administration irritation results show that no irritation is observed in the normal control group and the model control group, while relatively severe irritation caused by tail vein administration is observed in both the formulation 1 and the formulation 2, the irritation of the formulation 1 being greater than that of the formulation 2, and almost no irritation is observed with the formulation 3, which is significantly superior to the formulation 1 and the formulation 2 in terms of irritation.

It can be seen from the above results that, in the foreign body implantation malnutrition model rat test, the formulation 1, the formulation 2 and the formulation 3 have consistent trends in the aspect of the copper, manganese, zinc and selenium plasma concentrations of rats, all of which can significantly increase the copper, manganese, zinc and selenium plasma concentrations. Because the zinc concentration in the formulation 2 is significantly higher than that in the formulation 1 and the formulation 3, the zinc plasma concentrations of the rats given the formulation 2 are also significantly higher than those of the rats given the formulation 1 and the formulation 3, but all of them return to the normal control group level on the 10^{th} day. The formulation 2 and the formulation 3 are comparable in the aspect of reducing the white blood cell number of the model animals, namely, the formulation 2 and the formulation 3 are not obviously different in the aspect of the supplementation effects of various elements. However, in the aspect of irritation with repeated intravenous administration, the formulation 3 is superior to the formulation 1 and the formulation 2. For patients with severe clinical trace element requirements, such as patients with severe burn, multiple doses need to be used. The formulation 3, which has the advantage of low irritation with repeated administration, can better meet the safety requirement of clinical application.

The above-mentioned examples only express several embodiments of the present application, and their descriptions are relatively specific and detailed, but should not be understood as limiting the scope of the present application. It should be noted that various changes and modifications can be made by those skilled in the art without departing from the spirit of the present application, all of which are within the scope of protection of the present application. Therefore, the scope of protection of the present application should be subject to the appended claims.

1. A trace element composition, wherein it comprises a main material and an auxiliary material, wherein the main material consists of medicinal organic acid salt of ferrum element, medicinal organic acid salt of zinc element, medicinal organic acid salt of copper element, medicinal organic acid salt of manganese element, medicinal salt of fluorine element, medicinal salt of iodine element, medicinal salt of selenium element, medicinal salt of molybdenum element, medicinal organic acid salt or medicinal inorganic salt of chromium element, the auxiliary material consists of water for injection and a pH regulator, wherein the pH regulator is an inorganic acid and an optional inorganic base, the pH value of the trace element composition is 2.0 to 3.5, and in each 10 mL of the trace element composition, each trace element in the main material meets the following dosage: 17.8 to 21.5 μmol of ferrum, 40 to 100 μmol of zinc, 4.7 to 9.6 μmol of copper, 1 μmol of manganese, 25 to 60 μmol of fluorine, 0.5 to 1.2 μmol of iodine, 0.75 to 1.27 μmol of selenium, 0.2 to 0.26 μmol of molybdenum and 0.2 to 0.4 μmol of chromium.

2. The trace element composition as claimed in claim 1, wherein in each 10 mL of the trace element composition, each trace element in the main material meets the following dosage: 18 to 21.5 μmol of ferrum, 50 to 100 μmol of zinc, 4.7 to 6 μmol of copper, 1 μmol of manganese, 50 μmol of fluorine, 0.5 to 1.0 μmol of iodine, 1.0 to 1.25 μmol of selenium, 0.2 μmol of molybdenum and 0.2 μmol of chromium.

3. The trace element composition as claimed in claim 1, wherein in each 10 mL of the trace element composition, each trace element in the main material meets the following dosage: 20 μmol of ferrum, 77 μmol of zinc, 4.7 μmol or 6 μmol of copper, 1 μmol of manganese, 50 μmol of fluorine, 1 μmol of iodine, 1 μmol of selenium, 0.2 μmol of molybdenum and 0.2 μmol of chromium.

4. The trace element composition as claimed in claim 1, wherein the pH value of the composition is 2.6 to 3.2.

5. The trace element composition as claimed in claim 1, wherein the inorganic acid is hydrochloric acid or sulfuric acid, preferably hydrochloric acid; the inorganic base is sodium hydroxide or potassium hydroxide, preferably sodium hydroxide.

6. The trace element composition as claimed in claim 1, wherein the organic acid salt of ferrum element is selected from any one of ferrous gluconate, ferrous L-aspartate, ferrous DL-aspartate and ferrous fumarate, the organic acid salt of zinc element is selected from any one of zinc gluconate, zinc L-aspartate and zinc DL-aspartate, the organic acid salt of copper element is selected from any one of copper gluconate, copper L-aspartate and copper DL-aspartate, the organic acid salt of manganese element is selected from any one of manganese gluconate, manganese L-aspartate and manganese DL-aspartate, the medicinal salt of fluorine element is sodium fluoride or potassium fluoride, the medicinal salt of iodine element is sodium iodide or potassium iodide, the medicinal salt of selenium element is sodium selenite or selenous acid, the medicinal salt of molybdenum element is sodium molybdate or ammonium heptamolybdate, the medicinal inorganic salt of chromium element is chromium chloride or chromium sulfate, and/or the organic acid salt of chromium element is selected from any one of chromium gluconate, chromium L-aspartate and chromium DL-aspartate,
preferably, the main material consists of ferrous gluconate, zinc gluconate, copper gluconate, manganese gluconate, sodium fluoride, potassium iodide or sodium iodide, sodium selenite or sodium selenate, sodium molybdate and chromium chloride.

7. The trace element composition as claimed in claim 1, wherein the trace element composition is an injection; preferably, the inner wall of the packaging material of the injection is made of non-glass packaging material, and further preferably, the inner wall of the packaging material is made of polypropylene material.

8. A preparation method of the trace element composition as claimed in claim 1, comprising the steps of: dispersing salt of each trace element in injection water, adjusting to a target pH value by using a pH regulator, filtering to obtain a filtrate, adding water to the filtrate to full volume, potting, and sterilizing, wherein sterilizing is preferably performed using a terminal sterilization process, the F₀ value of the terminal sterilization process is ≥8, and preferably ≥12.

9. The preparation method as claimed in claim 8, wherein the packaging material in direct contact with the medicine during the potting is a non-glass packaging material, and preferably a polypropylene material;
preferably, the trace element composition is prepared under the condition of being exposed to no higher than 500Lux illumination, preferably no higher than 300Lux illumination, and more preferably no higher than 100Lux illumination.

10. A method for preventing and treating trace element deficiency comprising administering an effective amount of the trace element composition as claimed in claim 1.

11. The trace element composition as claimed in claim 2, wherein the pH value of the composition is 2.6 to 3.2.

12. The trace element composition as claimed in claim 3, wherein the pH value of the composition is 2.6 to 3.2.

13. The trace element composition as claimed in claim 2, wherein the inorganic acid is hydrochloric acid or sulfuric acid, preferably hydrochloric acid; the inorganic base is sodium hydroxide or potassium hydroxide, preferably sodium hydroxide.

14. The trace element composition as claimed in claim 3, wherein the inorganic acid is hydrochloric acid or sulfuric acid, preferably hydrochloric acid; the inorganic base is sodium hydroxide or potassium hydroxide, preferably sodium hydroxide.

15. The trace element composition as claimed in claim 4, wherein the inorganic acid is hydrochloric acid or sulfuric acid, preferably hydrochloric acid; the inorganic base is sodium hydroxide or potassium hydroxide, preferably sodium hydroxide.

16. The trace element composition as claimed in claim 2, wherein the organic acid salt of ferrum element is selected from any one of ferrous gluconate, ferrous L-aspartate, ferrous DL-aspartate and ferrous fumarate, the organic acid salt of zinc element is selected from any one of zinc gluconate, zinc L-aspartate and zinc DL-aspartate, the organic acid salt of copper element is selected from any one of copper gluconate, copper L-aspartate and copper DL-aspartate, the organic acid salt of manganese element is selected from any one of manganese gluconate, manganese L-aspartate and manganese DL-aspartate, the medicinal salt of fluorine element is sodium fluoride or potassium fluoride, the medicinal salt of iodine element is sodium iodide or potassium iodide, the medicinal salt of selenium element is sodium selenite or selenous acid, the medicinal salt of molybdenum element is sodium molybdate or ammonium heptamolybdate, the medicinal inorganic salt of chromium element is chromium chloride or chromium sulfate, and/or the organic acid salt of chromium element is selected from any one of chromium gluconate, chromium L-aspartate and chromium DL-aspartate,
preferably, the main material consists of ferrous gluconate, zinc gluconate, copper gluconate, manganese gluconate, sodium fluoride, potassium iodide or sodium iodide, sodium selenite or sodium selenate, sodium molybdate and chromium chloride.

17. The trace element composition as claimed in claim 3, wherein the organic acid salt of ferrum element is selected from any one of ferrous gluconate, ferrous L-aspartate, ferrous DL-aspartate and ferrous fumarate, the organic acid salt of zinc element is selected from any one of zinc gluconate, zinc L-aspartate and zinc DL-aspartate, the organic acid salt of copper element is selected from any one of copper gluconate, copper L-aspartate and copper DL-aspartate, the organic acid salt of manganese element is selected from any one of manganese gluconate, manganese L-aspartate and manganese DL-aspartate, the medicinal salt of fluorine element is sodium fluoride or potassium fluoride, the medicinal salt of iodine element is sodium iodide or potassium iodide, the medicinal salt of selenium element is sodium selenite or selenous acid, the medicinal salt of molybdenum element is sodium molybdate or ammonium heptamolybdate, the medicinal inorganic salt of chromium element is chromium chloride or chromium sulfate, and/or the organic acid salt of chromium element is selected from any one of chromium gluconate, chromium L-aspartate and chromium DL-aspartate,
preferably, the main material consists of ferrous gluconate, zinc gluconate, copper gluconate, manganese gluconate, sodium fluoride, potassium iodide or sodium iodide, sodium selenite or sodium selenate, sodium molybdate and chromium chloride.

18. The trace element composition as claimed in claim 4, wherein the organic acid salt of ferrum element is selected from any one of ferrous gluconate, ferrous L-aspartate, ferrous DL-aspartate and ferrous fumarate, the organic acid salt of zinc element is selected from any one of zinc gluconate, zinc L-aspartate and zinc DL-aspartate, the organic acid salt of copper element is selected from any one of copper gluconate, copper L-aspartate and copper DL-aspartate, the organic acid salt of manganese element is selected from any one of manganese gluconate, manganese L-aspartate and manganese DL-aspartate, the medicinal salt of fluorine element is sodium fluoride or potassium fluoride, the medicinal salt of iodine element is sodium iodide or potassium iodide, the medicinal salt of selenium element is sodium selenite or selenous acid, the medicinal salt of molybdenum element is sodium molybdate or ammonium heptamolybdate, the medicinal inorganic salt of chromium element is chromium chloride or chromium sulfate, and/or the organic acid salt of chromium element is selected from any one of chromium gluconate, chromium L-aspartate and chromium DL-aspartate,
preferably, the main material consists of ferrous gluconate, zinc gluconate, copper gluconate, manganese gluconate, sodium fluoride, potassium iodide or sodium iodide, sodium selenite or sodium selenate, sodium molybdate and chromium chloride.

19. The trace element composition as claimed in claim 5, wherein the organic acid salt of ferrum element is selected from any one of ferrous gluconate, ferrous L-aspartate, ferrous DL-aspartate and ferrous fumarate, the organic acid salt of zinc element is selected from any one of zinc gluconate, zinc L-aspartate and zinc DL-aspartate, the organic acid salt of copper element is selected from any one of copper gluconate, copper L-aspartate and copper DL-aspartate, the organic acid salt of manganese element is selected from any one of manganese gluconate, manganese L-aspartate and manganese DL-aspartate, the medicinal salt of fluorine element is sodium fluoride or potassium fluoride, the medicinal salt of iodine element is sodium iodide or potassium iodide, the medicinal salt of selenium element is sodium selenite or selenous acid, the medicinal salt of molybdenum element is sodium molybdate or ammonium heptamolybdate, the medicinal inorganic salt of chromium element is chromium chloride or chromium sulfate, and/or the organic acid salt of chromium element is selected from any one of chromium gluconate, chromium L-aspartate and chromium DL-aspartate,
preferably, the main material consists of ferrous gluconate, zinc gluconate, copper gluconate, manganese gluconate, sodium fluoride, potassium iodide or sodium iodide, sodium selenite or sodium selenate, sodium molybdate and chromium chloride.

20. The trace element composition as claimed in claim 2, wherein the trace element composition is an injection; preferably, the inner wall of the packaging material of the injection is made of non-glass packaging material, and further preferably, the inner wall of the packaging material is made of polypropylene material.
